(19) United States
(12) Patent Application Publication
(10) Pub. No.: US 2018/0002247 A1
(43) Pub. Date: Jan. 4, 2018

(54) METHODS FOR CHIRAL RESOLUTION OF TROLOX

(71) Applicant: BIOELECTRON TECHNOLOGY CORPORATION, Mountain View, CA (US)

(72) Inventors: Paul MOLLARD, Mountain View, CA (US); Peter GIANNOUSIS, Mountain View, CA (US); Mahmoud MIRMEHRABI, Halifax (CA); Hekla ALITE, Devens, MA (US); Aniruddh SINGH, Devens, MA (US)

(21) Appl. No.: 15/536,514

(22) PCT Filed: Dec. 16, 2015
(86) PCT No.: PCT/US2015/066208

§ 371 Date: Jun. 15, 2017

Related U.S. Application Data
(60) Provisional application No. 62/092,743, filed on Dec. 16, 2014.
(60) Provisional application No. 62/133,276, filed on Mar. 13, 2015.

(51) Int. Cl. C07B 57/00; C07C 51/487; C07D 311/66

(52) U.S. Cl. CPC C07B 57/00; C07D 311/66; C07C 51/487; C07B 2200/13; C07B 2200/07

(57) ABSTRACT

The invention relates to methods of separating Trolox isomers (R)-Trolox and (S)-Trolox, comprising: (a) contacting a mixture of (R) and (S)-Trolox with a resolving agent selected from the group consisting of (1S,2S)-(+)-Pseudoephedrine, (R)-(+)-2-Amino-3-phenyl-1-propanol, (1R,2R)-(−)-Pseudoephedrine, and (S)-(−)-2-Amino-3-phenyl-1-propanol, wherein the resolving agent forms a solid salt with one of (R)-Trolox and (S)-Trolox, and substantially does not form a solid salt with the other; and (b) separating the solid salt from the Trolox isomer that did not form the solid salt with the resolving agent.

The application claims priority to, and the benefit of, U.S. Provisional Patent Application No. 62/092,743, filed Dec. 16, 2014, entitled POLYMORPHIC AND AMORPHOUS FORMS OF (R)-2-HYDROXY-2-METHYL-4-(2,4,5-TRIMETHYL-3,6-DIOXOCYCLOHEXA-1,4-DIENYL)BUTANAMIDE, and U.S. Provisional Patent Application No. 62/133,276, filed Mar. 13, 2015, entitled POLYMORPHIC AND AMORPHOUS FORMS OF (R)-2-HYDROXY-2-METHYL-4-(2,4,5-TRIMETHYL-3,6-DIOXOCYCLOHEXA-1,4-DIENYL)BUTANAMIDE, the contents of both of which are herein incorporated by reference in their entirety for all purposes.

### BACKGROUND

PCT Application No. PCT/US2008/082374 describes a synthesis for racemic 2-hydroxy-2-methyl-4-(2,4,5-trimethyl-3,6-dioxocyclohexa-1,4-dienyl)butanamide, which is useful for treating and/or suppressing mitochondrial disorders and certain pervasive developmental disorders, from racemic Trolox (6-hydroxy-2,5,7,8-tetramethylchroman-2-carboxylic acid).

Chiral resolving agents may be useful in separating enantiomers. For example, a chiral resolving agent may form a solid salt with one enantiomer, but not with the other enantiomer (which remains in solution or as an oil); the two enantiomers may then be separated by filtering the solid. However, not all resolving agents are useful for separating the enantiomers of a particular compound. Furthermore, resolving agents differ in their ability to provide, for example, better resolution, higher yields, easier scale up, and/or improved ease of use.

Racemic Trolox has been previously resolved into its (R) and (S)-isomers with α-methyl benzyl amine (MBA) and R-(+)-N-Benzyl-α-phenylethylamine resolving agents. See, for example, U.S. Pat. Nos. 3,947,473, 4,003,919, and 4,026,907, and U.S. Patent Application Publication No. 2011/0251407.

What is needed are improved methods, reagents, and reagent mixtures and combinations, for synthesizing particular stereoisomers of 2-hydroxy-2-methyl-4-(2,4,5-trimethyl-3,6-dioxocyclohexa-1,4-dienyl)butanamide, such as (R)-2-hydroxy-2-methyl-4-(2,4,5-trimethyl-3,6-dioxocyclohexa-1,4-dienyl)butanamide and (S)-2-hydroxy-2-methyl-4-(2,4,5-trimethyl-3,6-dioxocyclohexa-1,4-dienyl)butanamide. In particular, what is needed are improved methods for obtaining specific Trolox enantiomers in high purity.

### BRIEF SUMMARY OF THE INVENTION

In one aspect of the invention is a method of separating Trolox isomers (R)-Trolox and (S)-Trolox, comprising: (a) contacting a mixture of (R) and (S)-Trolox with a resolving agent selected from the group consisting of N-methyl-D-glucamine, L-Arginine, L-Lysine, (1S,2S)-(+)-Pseudoephedrine, (R)-(−)-Leucinol, D-Lysine, (R)-(+)-2-Amino-3-phenyl-1-propanol, (1R,2R)-(−)-Pseudoephedrine, and (S)-(+2-Amino-3-phenyl-1-propanol, wherein a solid salt forms between the resolving agent and only one of (R)-Trolox and (S)-Trolox, and substantially does not form a solid salt with the other; and (b) separating the solid salt from the Trolox isomer that did not form the solid salt with the resolving agent. In some embodiments, the resolving agent is (1S,2S)-(+)-Pseudoephedrine. In some embodiments, the resolving agent is (R)-(+)-2-Amino-3-phenyl-1-propanol. In some embodiments, the resolving agent is (1R,2R)-(−)-Pseudoephedrine. In some embodiments, the resolving agent is and (S)-(−)-2-Amino-3-phenyl-1-propanol. In some embodiments, the resolving agent is N-methyl-D-glucamine. In some embodiments, the resolving agent is L-Arginine. In some embodiments, the resolving agent is L-Lysine. In some embodiments, the resolving agent is (R)-(−)-Leucinol. In some embodiments, the resolving agent is D-Lysine. In some embodiments, the method is a method of separating Trolox isomers (R)-Trolox and (S)-Trolox, comprising: (a) contacting a mixture of (R) and (S)-Trolox with a resolving agent selected from the group consisting of (1S,2S)-(+)-Pseudoephedrine, (R)-(+)-2-Amino-3-phenyl-1-propanol, (1R,2R)-(−)-Pseudoephedrine, and (S)-(−)-2-Amino-3-phenyl-1-propanol wherein the resolving agent forms a solid salt with one of (R)-Trolox and (S)-Trolox, and substantially does not form a solid salt with the other; and (b) separating the solid salt from the Trolox isomer that did not form the solid salt with the resolving agent. In some embodiments, step (a) comprises dissolving the mixture of (R) and (S)-Trolox and the resolving agent in a solvent. In some embodiments, including any of the foregoing embodiments, step (a) comprises: (i) heating the mixture of (R) and (S)-Trolox and the resolving agent in the solvent until dissolution occurs; and (ii) cooling the mixture from (i). In some embodiments, including any of the foregoing embodiments, the heating in step (a)(i) comprises heating to reflux temperature. In some embodiments, including any of the foregoing embodiments, the cooling in step (a)(ii) occurs over at least about one hour. In some embodiments, including any of the foregoing embodiments, the cooling in step (a)(ii) occurs over at least about two hours. In some embodiments, including any of the foregoing embodiments, the cooling in step (a)(ii) occurs over at least about eight hours. In some embodiments, including any of the foregoing embodiments, the cooling in step (a)(ii) comprises cooling to about 0° C. to about 30° C. In some embodiments, including any of the foregoing embodiments, the cooling in step (a)(ii) comprises cooling to about 20° C. to about 30° C. In some embodiments, including any of the foregoing embodiments, the cooling in step (a)(ii) comprises cooling to about 20° C. to about 26° C. In some embodiments, including any of the foregoing embodiments, the solvent is a polar solvent. In some embodiments, including any of the foregoing embodiments, the solvent is ethyl acetate. In some embodiments, including any of the foregoing embodiments, the solvent is isopropyl acetate. In some embodiments, including any of the foregoing embodiments, the solvent is ethyl acetate with 1% water. In some embodiments, including any of the foregoing embodiments, the solvent is 2-methyltetrahydrofuran. In some embodiments, including any of the foregoing embodiments, about 3 to about 7 volumes of solvent are added in step (a). In some embodiments, including any of the foregoing embodiments, about 4 to about 6 volumes of solvent are added in step (a). In various embodiments, including any of the foregoing embodiments, about 3 volumes of solvent, about 4 volumes of solvent, about 5 volumes of solvent, about 6 volumes of solvent, or about 7 volumes of solvent are added in step (a). In some embodiments, including any of the foregoing embodiments, about 0.50 to about 2.0 equivalents of resolving agent are used in step (a). In some embodiments, including any of the foregoing embodiments, about 0.60 to about 1.30 equivalents of resolving agent are used in step (a). In some embodiments, including any of the foregoing embodiments, about 0.80 to about 1.30 equivalents of resolving agent are used in step (a). In some embodiments, including any of the foregoing embodiments, about 0.95 to about 1.20 equivalents of resolving agent are used in step (a). In some embodiments, including any of the foregoing embodiments, about 1.05 equivalents of resolving agent are used in step (a). In some embodiments, including any of the foregoing embodiments, about 1.15 equivalents of resolving agent are used in step (a). In various embodiments, including any of the foregoing embodiments, about 0.5 to about 2 equivalents, about 0.6 to about 1.5 equivalents, about 0.9 to about 1.5 equivalents, about 1.00 to about 1.10 equivalents, about 0.65 to about 1.25 equivalents, about 0.50 to about 0.60 equivalents, about 0.60 to about 0.70 equivalents, about 0.80 to about 0.90 equivalents, about 0.80 to about 1.30 equivalents, about 0.85 to about 1.25 equivalents, about 0.95 to about 1.05 equivalents, about 0.95 to about 1.20 equivalents, about 0.95 to about 1.20 equivalents, about 1.10 to about 1.20 equivalents, about 1.20 to about 1.30 equivalents, about 0.50 to about 0.60 equivalents, about 0.50 equivalents, about 0.55 equivalents, about 0.60 equivalents, about 0.65 equivalents, about 0.70 equivalents, about 0.75 equivalents, about 0.80 equivalents, about 0.85 equivalents, about 0.90 equivalents, about 0.95 equivalents, about 1.00 equivalents, about 1.05 equivalents, about 1.10 equivalents, about 1.15 equivalents, about 1.20 equivalents, about 1.25 equivalents, or about 1.30 equivalents of resolving agent are used in step (a). In some embodiments, including any of the foregoing embodiments, step (a) comprises: (i) evaporating any solvents present, and (ii) adding diethyl ether to the mixture. In some embodiments, including any of the foregoing embodiments, the diethyl ether is removed. In some embodiments, including any of the foregoing embodiments, the mixture in step (a) is seeded with the desired solid salt. In some embodiments, including any of the foregoing embodiments, step (b) comprises filtering the solid salt. In some embodiments, including any of the foregoing embodiments, step (b) further comprises a step (b)(1), comprising slurrying the solid salt in the solvent. In some embodiments, including any of the foregoing embodiments, step (b) and/or step (b)(1) further comprise rinsing and drying the solid salt. In some embodiments, including any of the foregoing embodiments, the method further comprises a step (c): (c) separating the Trolox isomer contained in the solid salt from the resolving agent. In some embodiments, including any of the foregoing embodiments, step (c) comprises adding an acid to the solid salt. In some embodiments, including any of the foregoing embodiments, step (c) comprises adding a base to the solid salt. In some embodiments, including any of the foregoing embodiments, the resolving agent is (1S,2S)-(+)-Pseudoephedrine. In some embodiments, including any of the foregoing embodiments, the resolving agent is (R)-(+)-2-Amino-3-phenyl-1-propanol. In some embodiments, including any of the foregoing embodiments, the resolving agent is (1R,2R)-(1)-Pseudoephedrine. In some embodiments, including any of the foregoing embodiments, the resolving agent is (S)-(−)-2-Amino-3-phenyl-1-propanol. In some embodiments, including any of the foregoing embodiments, the Trolox isomer that forms the solid salt with the resolving agent is (R)-Trolox. In some embodiments, including any of the foregoing embodiments, the Trolox isomer that forms the solid salt with the resolving agent is (S)-Trolox. In some embodiments, including any of the foregoing embodiments, the mixture of (R)-Trolox and (S)-Trolox is a racemic mixture.

In some embodiments, including any of the foregoing embodiments in the preceding paragraph, the Trolox isomer that forms the solid salt with the resolving agent is (R)-Trolox. In some embodiments, including any of the foregoing embodiments, the resolving agent is (1S,2S)-(+)-Pseudoephedrine. In some embodiments, including any of the foregoing embodiments, the resolving agent is (R)-(+)-2-Amino-3-phenyl-1-propanol. In some embodiments, the method comprises: (1) contacting a mixture of (R)-Trolox and (S)-Trolox with about 0.8 to about 1.30 equivalents of (1S,2S)-(+)-Pseudoephedrine and ethyl acetate; (2) heating the mixture until dissolution is achieved; (3) cooling the mixture to about 20° C. to about 30° C. over at least about 50 minutes; (4) cooling the mixture to about 5° C. to about 15° C.; (7) filtering the resulting slurry; (8) washing the wet cake with ethyl acetate; (9) and drying the solids. In some embodiments, the method comprises: (1) contacting a mixture of (R)-Trolox and (S)-Trolox with about 1.10 to about 1.20 equivalents of (1S,2S)-(+)-Pseudoephedrine and about 4 to about 6 volumes of ethyl acetate; (2) heating the mixture to between about 35° C. to about 55° C. until dissolution is achieved; (3) cooling the mixture to about 20° C. to about 30° C. over at least about 50 minutes; (4) cooling the mixture to about 5° C. to about 15° C. over about 20 to about 40 minutes; (5) holding the temperature in step (4) for about 50-70 minutes; (7) filtering the resulting slurry; (8) washing the wet cake with about 5 to about 7 volumes of ethyl acetate at room temperature; (9) and drying the solids. In some embodiments, the method comprises: (1) contacting a mixture of (R)-Trolox and (S)-Trolox with about 1.15 equivalents of (1S,2S)-(+)-Pseudoephedrine and about 5 volumes of ethyl acetate; (2) heating the mixture to between about 40° C. to about 50° C. until dissolution is achieved; (3) cooling the mixture to room temperature over at least about 50 minutes; (4) cooling the mixture to about 10° C. over about 30 minutes; (5) holding the temperature in step (4) for about 50-70 minutes; (7) filtering the resulting slurry; (8) washing the wet cake with about 6 volumes of ethyl acetate at room temperature; (9) and drying the solids. In some embodiments, including any of the foregoing embodiments, the enantiomeric excess of the (R)-Trolox obtained from the method is at least about 98%. In some embodiments, including any of the foregoing embodiments, the enantiomeric excess of the (R)-Trolox or salt thereof obtained from the method is at least about 99%. In some embodiments, including any of the foregoing embodiments, the enantiomeric excess of the (R)-Trolox or salt thereof obtained from the method is at least about 99.5%. In some embodiments, including any of the foregoing embodiments, the enantiomeric excess of the (R)-Trolox or salt thereof obtained from the method is at least about 99.9%. In various embodiments, including any of the foregoing embodiments, the enantiomeric excess of the (R)-Trolox or salt thereof obtained from the method is at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%. In various embodiments, including any of the foregoing embodiments, the yield of the (R)-Trolox or salt thereof obtained from the method is at least about 50%, at least about 55%, at least about 60%, at least about 65%, at least about 70%, at least about 75%, at least about 80%, or at least about 85%. In various embodiments, including any of the foregoing embodiments, the purity of the (R)-Trolox or salt thereof obtained from the method, exclusive of any solvents, carriers or excipients, is at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%. In some embodiments, the yield of the (R)-Trolox or salt thereof is at least about 50%, the enantiomeric excess is at least about 97%, and the purity is at least about 99%. In some embodiments, the yield of the (R)-Trolox or salt thereof is at least about 70%, the enantiomeric excess is at least about 98%, and the purity is at least about 99%. In some embodiments, the yield of the (R)-Trolox or salt thereof is at least about 75%, the enantiomeric excess is at least about 99%, and the purity is at least about 99%. In some embodiments, the yield of the (R)-Trolox or salt thereof is at least about 80%, the enantiomeric excess is at least about 99.5%, and the purity is at least about 99%. In various embodiments, including any of the foregoing embodiments, the (R)-Trolox or salt thereof that is obtained from the method is converted to (R)-2-hydroxy-2-methyl-4-(2,4,5-trimethyl-3,6-dioxocyclohexa-1,4-dienyl)butanamide or a salt thereof.

In some embodiments, including any of the foregoing embodiments in the paragraph preceding the above paragraph, the Trolox isomer that forms the solid salt with the resolving agent is (S)-Trolox. In some embodiments, including any of the foregoing embodiments, the resolving agent is (1R,2R)-(−)-Pseudoephedrine. In some embodiments, including any of the foregoing embodiments, the resolving agent is (S)-(−)-2-Amino-3-phenyl-1-propanol. In some embodiments, including any of the foregoing embodiments, the enantiomeric excess of the (S)-Trolox or salt thereof obtained from the method is at least about 98%. In some embodiments, including any of the foregoing embodiments, the enantiomeric excess of the (S)-Trolox or salt thereof obtained from the method is at least about 99%. In some embodiments, including any of the foregoing embodiments, the enantiomeric excess of the (S)-Trolox or salt thereof obtained from the method is at least about 99.5%. In some embodiments, including any of the foregoing embodiments, the enantiomeric excess of the (S)-Trolox or salt thereof obtained from the method is at least about 99.9%. In various embodiments, including any of the foregoing embodiments, the enantiomeric excess of the (S)-Trolox or salt thereof obtained from the method is at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%. In various embodiments, including any of the foregoing embodiments, the yield of the (S)-Trolox or salt thereof obtained from the method is at least about 50%, at least about 55%, at least about 60%, at least about 65%, at least about 70%, at least about 75%, at least about 80%, or at least about 85%. In various embodiments, including any of the foregoing embodiments, the purity of the (S)-Trolox or salt thereof obtained from the method, exclusive of any solvents, carriers or excipients, is at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%. In some embodiments, the yield of the (S)-Trolox or salt thereof is at least about 50%, the enantiomeric excess is at least about 97%, and the purity is at least about 99%. In some embodiments, the yield of the (S)-Trolox or salt thereof is at least about 70%, the enantiomeric excess is at least about 98%, and the purity is at least about 99%. In some embodiments, the yield of the (S)-Trolox or salt thereof is at least about 75%, the enantiomeric excess is at least about 99%, and the purity is at least about 99%. In some embodiments, the yield of the (S)-Trolox or salt thereof is at least about 80%, the enantiomeric excess is at least about 99.5%, and the purity is at least about 99%.

In another aspect of the invention is (R)-Trolox or salt thereof, produced according to a method described herein. In various embodiments, the enantiomeric excess of the (R)-Trolox or salt thereof is at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%. In various embodiments, including any of the foregoing embodiments, the yield of the (R)-Trolox or salt thereof is at least about 50%, at least about 55%, at least about 60%, at least about 65%, at least about 70%, at least about 75%, at least about 80%, at least about 85%. In various embodiments, including any of the foregoing embodiments, the purity of the (R)-Trolox or salt thereof, exclusive of any solvents, carriers or excipients, is at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%. In some embodiments, the yield of the (R)-Trolox or salt thereof is at least about 50%, the enantiomeric excess is at least about 97%, and the purity is at least about 99%. In some embodiments, the yield of the (R)-Trolox or salt thereof is at least about 70%, the enantiomeric excess is at least about 98%, and the purity is at least about 99%. In some embodiments, the yield of the (R)-Trolox or salt thereof is at least about 75%, the enantiomeric excess is at least about 99%, and the purity is at least about 99%. In some embodiments, the yield of the (R)-Trolox or salt thereof is at least about 80%, the enantiomeric excess is at least about 99.5%, and the purity is at least about 99%.

In another aspect of the invention is (S)-Trolox or salt thereof, produced according to a method described herein. In various embodiments, the enantiomeric excess of the (S)-Trolox or salt thereof is at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%. In various embodiments, including any of the foregoing embodiments, the yield of the (S)-Trolox or salt thereof is at least about 50%, at least about 55%, at least about 60%, at least about 65%, at least about 70%, at least about 75%, at least about 80%, at least about 85%. In various embodiments, including any of the foregoing embodiments, the purity of the (S)-Trolox or salt thereof, exclusive of any solvents, carriers or excipients, is at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%. In some embodiments, the yield of the (S)-Trolox or salt thereof is at least about 50%, the enantiomeric excess is at least about 97%, and the purity is at least about 99%. In some embodiments, the yield of the (S)-Trolox or salt thereof is at least about 70%, the enantiomeric excess is at least about 98%, and the purity is at least about 99%. In some embodiments, the yield of the (S)-Trolox or salt thereof is at least about 75%, the enantiomeric excess is at least about 99%, and the purity is at least about 99%. In some embodiments, the yield of the (S)-Trolox or salt thereof is at least about 80%, the enantiomeric excess is at least about 99.5%, and the purity is at least about 99%.

In another aspect of the invention is converting the (R)-Trolox or salt thereof that is obtained from any method described herein to (R)-2-hydroxy-2-methyl-4-(2,4,5-trimethyl-3,6-dioxocyclohexa-1,4-dienyl)butanamide or a salt thereof.

In another aspect of the invention is converting the (S)-Trolox or salt thereof that is obtained from any method described herein to (S)-2-hydroxy-2-methyl-4-(2,4,5-trimethyl-3,6-dioxocyclohexa-1,4-dienyl)butanamide or a salt thereof.

In another aspect of the invention is the compound (R)-Trolox (1S,2S)-(+)-Pseudoephedrine salt. In some embodiments, the compound is a 1:1 salt.

In another aspect of the invention is the compound (R)-Trolox (R)-(+)-2-Amino-3-phenyl-1-propanol salt. In some embodiments, the compound is a 1:1 salt.

In another aspect of the invention is the compound (S)-Trolox (1R,2R)-(−)-Pseudoephedrine salt.

In some embodiments, the compound is a 1:1 salt.

In another aspect of the invention is the compound (S)-Trolox (S)-(−)-2-Amino-3-phenyl-1-propanol salt. In some embodiments, the compound is a 1:1 salt.

In another aspect of the invention is (R)-2-hydroxy-2-methyl-4-(2,4,5-trimethyl-3,6-dioxocyclohexa-1,4-dienyl)butanamide or the hydroquinone form thereof, or salt thereof, produced according to a method described herein. In some embodiments, is (R)-2-hydroxy-2-methyl-4-(2,4,5-trimethyl-3,6-dioxocyclohexa-1,4-dienyl)butanamide or salt thereof, produced according to a method described herein.

In another aspect of the invention is a pharmaceutical composition comprising (R)-2-hydroxy-2-methyl-4-(2,4,5-trimethyl-3,6-dioxocyclohexa-1,4-dienyl)butanamide or the hydroquinone form thereof as produced by a method described herein, or a pharmaceutically acceptable salt thereof, and a pharmaceutically acceptable carrier. In some embodiments, the pharmaceutical composition comprises (R)-2-hydroxy-2-methyl-4-(2,4,5-trimethyl-3,6-dioxocyclohexa-1,4-dienyl)butanamide as produced by a method described herein, or a pharmaceutically acceptable salt thereof, and a pharmaceutically acceptable carrier.

In another aspect of the invention is (S)-2-hydroxy-2-methyl-4-(2,4,5-trimethyl-3,6-dioxocyclohexa-1,4-dienyl)butanamide or the hydroquinone form thereof, or salt thereof, produced according to a method described herein. In some embodiments, is (S)-2-hydroxy-2-methyl-4-(2,4,5-trimethyl-3,6-dioxocyclohexa-1,4-dienyl)butanamide or salt thereof, produced according to a method described herein.

In another aspect of the invention is a pharmaceutical composition comprising (S)-2-hydroxy-2-methyl-4-(2,4,5-trimethyl-3,6-dioxocyclohexa-1,4-dienyl)butanamide or the hydroquinone form thereof as produced by a method described herein, or a pharmaceutically acceptable salt thereof, and a pharmaceutically acceptable carrier. In some embodiments, the pharmaceutical composition comprises (S)-2-hydroxy-2-methyl-4-(2,4,5-trimethyl-3,6-dioxocyclohexa-1,4-dienyl)butanamide as produced by a method described herein, or a pharmaceutically acceptable salt thereof, and a pharmaceutically acceptable carrier.

In another aspect of the invention is a method of treating or suppressing an oxidative stress disorder, comprising administering a pharmaceutical composition as described herein to a subject in need thereof In some embodiments, the method is a method of treating the oxidative stress disorder. In some embodiments, the method is a method of suppressing the oxidative stress disorder.

For all compositions described herein, and all methods using or making a composition described herein, the compositions and methods can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the synthetic method, but the method does not contain any other steps which substantially affect the synthetic method other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

### BRIEF DESCRIPTION OF THE FIGURES

FIG. 1 shows a DSC thermogram of R-Trolox (1S,2S)-(+)-Pseudoephedrine salt (sample 1957-57-6).

### DETAILED DESCRIPTION

The chiral resolving agents of the present invention resolve Trolox enantiomers with improved performance.

Applicants have resolved Trolox enantiomers with a double α-methylbenzylamine (MBA) resolution, wherein (S)-(−)-α-methylbenzylamine forms a salt with (S)-Trolox, which is removed to enrich the content of (R)-Trolox, and then subsequently wherein the mother liquor is acidified to remove excess (S)-(−)-α-methylbenzylamine, and subjected to a second resolution with (R)-(+)-α-methylbenzylamine, thus forming a salt with (R)-Trolox. However, this process involved multiple steps (e.g. resolving with the (S)-(−)-α-methylbenzylamine in one step, and then resolving with the (R)-(+)-α-methylbenzylamine in another step). Furthermore, the MBA double resolution resulted in reaction product consistencies that were more difficult to work with (e.g. a yogurt-thickness consistency for the (R)-Trolox-(R)-(+)-α-methylbenzyl amine salt), difficulty in removing liquid, difficulty in stirring the reaction, poor filtration, and difficulty in scale-up.

In contrast, the current method is a single step resolution (e.g. a single chiral resolving agent), and further, in various embodiments, the current method is easier to scale up, is easier to stir, provides readily filterable solids, and provides a higher resolution and/or purity of the desired product. The current method may in some embodiments not require a recrystallization step, in contrast with previous methods.

In contrast, in various embodiments, the current method is easier to scale up, is easier to stir, provides readily filterable solids, is a single step resolution (e.g. a single chiral resolving agent) and/or provides a higher resolution and/or purity of the desired product. The current method may in some embodiments not require a recrystallization step, in contrast with previous methods. The current method may further, in various embodiments, provide high purity of product and/or high resolution of product at a larger scale.

The abbreviations used herein have their conventional meaning within the chemical and biological arts, unless otherwise specified.

Reference to “about” a value or parameter herein includes (and describes) variations that are directed to that value or parameter per se. For example, description referring to “about X” includes description of “X”.

The terms “a” or “an,” as used in herein means one or more, unless context clearly dictates otherwise.

By “subject,” “individual,” or “patient” is meant an individual organism, preferably a vertebrate, more preferably a mammal, most preferably a human.

“Treating” a disease with the compounds and methods discussed herein is defined as administering one or more of the compounds discussed herein, with or without additional therapeutic agents, in order to reduce or eliminate either the disease or one or more symptoms of the disease, or to retard the progression of the disease or of one or more symptoms of the disease, or to reduce the severity of the disease or of one or more symptoms of the disease. “Suppression” of a disease with the compounds and methods discussed herein is defined as administering one or more of the compounds discussed herein, with or without additional therapeutic agents, in order to suppress the clinical manifestation of the disease, or to suppress the manifestation of adverse symptoms of the disease. The distinction between treatment and suppression is that treatment occurs after adverse symptoms of the disease are manifest in a subject, while suppression occurs before adverse symptoms of the disease are manifest in a subject. Suppression may be partial, substantially total, or total. Because many of the mitochondrial disorders are inherited, genetic screening can be used to identify patients at risk of the disease. The compounds and methods of the invention can then be administered to asymptomatic patients at risk of developing the clinical symptoms of the disease, in order to suppress the appearance of any adverse symptoms.

“Therapeutic use” of the compounds discussed herein is defined as using one or more of the compounds discussed herein to treat or suppress a disease, as defined above. A “therapeutically effective amount” of a compound is an amount of the compound, which, when administered to a subject, is sufficient to reduce or eliminate either a disease or one or more symptoms of a disease, or to retard the progression of a disease or of one or more symptoms of a disease, or to reduce the severity of a disease or of one or more symptoms of a disease, or to suppress the clinical manifestation of a disease, or to suppress the manifestation of adverse symptoms of a disease. A therapeutically effective amount can be given in one or more administrations.

Methods of the invention utilize a resolving agent to separate the (R)- and (S)-Trolox enantiomers, wherein the resolving agent forms a solid salt with one of (R)-Trolox and (S)-Trolox, and substantially does not form a solid salt with the other under the particular reaction conditions. In some embodiments, when the resolving agent forms a solid salt with a Trolox enantiomer, at least about 50% of that Trolox enantiomer forms the solid salt with the resolving agent under the particular reaction conditions. In various embodiments, when the resolving agent forms a solid salt with a Trolox enantiomer, at least about 60%, at least about 70%, at least about 80%, at least about 85%, at least about 90%, at least about 95%, at least about 96%, at least about 97%, at least about 98%, or at least about 99% of that Trolox enantiomer forms the solid salt with the resolving agent under the particular reaction conditions. By “substantially does not form a solid salt” indicates that less than about 10% of the (non-solid salt forming) Trolox enantiomer forms a solid salt with the resolving agent under the particular reaction conditions. In various embodiments, “substantially does not form a solid salt” indicates that less than about 9%, less than about 8%, less than about 7%, less than about 6%, less than about 5%, less than about 4%, less than about 3%, less than about 2%, less than about 1%, less than about 0.5%, or less than about 0.1% of the (non-solid salt forming) Trolox enantiomer forms a solid salt with the resolving agent under the particular reaction conditions. As a non-limiting example, in some embodiments (1S,2S)-(+)-Pseudoephedrine forms a solid salt with (R)-Trolox (e.g. in some embodiments, at least about 50%, at least about 60%, at least about 70%, at least about 80%, or at least about 90%, of the (R)-Trolox that is present forms a solid salt with the (1S,2S)-(+)-Pseudoephedrine, and the (1S,2S)-(+)-Pseudoephedrine substantially does not form a solid salt with the (S)-Trolox that is present under the particular reaction conditions (e.g. in some embodiments, less than about 10%, less than about 9%, less than about 8%, less than about 7%, less than about 6%, less than about 5%, less than about 4%, less than about 3%, less than about 2%, less than about 1%, less than about 0.5%, or less than about 0.1% of the (S)-Trolox that is present forms a solid salt with the (1S,2S)-(+)-Pseudoephedrine under the particular reaction conditions.

“Yield” indicates the % of Trolox enantiomer obtained, relative to amount of starting material. For example, if 100 g of a 50/50 racemic mixture of (R)/(S)-Trolox are resolved, and 50 g of (S)-Trolox is recovered, the yield would be 100%. If 30 g of (S)-Trolox are recovered, the yield would be 60%. With regards to recovery of a Trolox salt, the yield is calculated as though only the Trolox, and not the salt counterion, is present. For example, if 100 g of a 50/50 racemic mixture of (R)/(S)-Trolox are resolved, and 40 g of an (S)-Trolox salt is recovered, and if the theoretical weight of the (S)-Trolox contained within that salt is 30 g, then the yield would be 60%.

While the compounds described herein can occur and can be used as the neutral (non-salt) compound, the description is intended to embrace all salts of the compounds described herein, as well as methods of using such salts of the compounds. In one embodiment, the salts of the compounds comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N-dibenzylethylenediamine, and triethylamine salts. Salts of acidic compounds with amino acids, such as lysine salts, can also be prepared. Additional salts particularly useful for pharmaceutical preparations are described in Berge S. M. et al., “Pharmaceutical salts,” 1. Pharm. Sci. 1977 January; 66(1): 1-19.

The invention includes methods of separating Trolox isomers (R)-Trolox and (S)-Trolox, comprising: (a) contacting a mixture of (R) and (S)-Trolox with a resolving agent selected from the group consisting of N-methyl-D-glucamine, L-Arginine, L-Lysine, (1S,2S)-(+)-Pseudoephedrine, (R)-(−)-Leucinol, D-Lysine, (R)-(+)-2-Amino-3-phenyl-1-propanol, (1R,2R)-(−)-Pseudoephedrine, and (S)-(+2-Amino-3-phenyl-1-propanol, wherein a solid salt forms between the resolving agent and only one of (R)-Trolox and (S)-Trolox, and substantially does not form a solid salt with the other; and (b) separating the solid salt from the Trolox isomer that did not form the solid salt with the resolving agent. In some embodiments, the resolving agent is selected from the group consisting of (1S,2S)-(+)-Pseudoephedrine and (R)-(+)-2-Amino-3-phenyl-1-propanol. In some embodiments, the resolving agent is selected from the group consisting of (1R,2R)-(−)-Pseudoephedrine and (S)-(+2-Amino-3-phenyl-1-propanol. In some embodiments, the resolving agent is (1S,2S)-(+)-Pseudoephedrine. In some embodiments, the resolving agent is (R)-(+)-2-Amino-3-phenyl-1-propanol. In some embodiments, the resolving agent is (1R,2R)-(−)-Pseudoephedrine. In some embodiments, the resolving agent is (S)-(−)-2-Amino-3-phenyl-1-propanol. In some embodiments, the resolving agent is N-methyl-D-glucamine. In some embodiments, the resolving agent is L-Arginine. In some embodiments, the resolving agent is L-Lysine. In some embodiments, the resolving agent is (R)-(−)-Leucinol. In some embodiments, the resolving agent is D-Lysine. In some embodiments, the mixture of (R)-Trolox and (S)-Trolox is a racemic mixture.

In some embodiments, step (a) comprises dissolving the mixture of (R)-Trolox and (S)-Trolox and the resolving agent in a solvent. In some embodiments, step (a) comprises: (i) heating the mixture of (R)-Trolox and (S)-Trolox and the resolving agent in the solvent until dissolution occurs; and (ii) cooling the mixture from (i). In some embodiments, dissolution indicates complete dissolution of all (R)-Trolox, (S)-Trolox, and resolving agent present in the mixture. In some embodiments, the heating in step (a)(i) comprises heating to reflux temperature. In some embodiments, the cooling in step (a)(ii) occurs over at least about one hour. In some embodiments, the cooling in step (a)(ii) occurs over at least about two hours. In some embodiments, the cooling in step (a)(ii) occurs over at least about eight hours. In some embodiments, the cooling in step (a)(ii) comprises cooling to about 0° C. to about 30° C. In some embodiments, the cooling in step (a)(ii) comprises cooling to about 20° C. to about 30° C. In some embodiments, the cooling in step (a)(ii) comprises cooling to about 20° C. to about 26° C.

In some embodiments, the solvent is a polar solvent. In some embodiments, the solvent is ethyl acetate. In some embodiments, the solvent is isopropyl acetate. In some embodiments, the solvent is ethyl acetate with 1% water. In some embodiments, the solvent is 2-methyltetrahydrofuran. In some embodiments, about 3 to about 7 volumes of solvent are added in step (a). In some embodiments, about 4 to about 6 volumes of solvent are added in step (a). In some embodiments, about 3 volumes of solvent are added in step (a). In some embodiments, about 4 volumes of solvent are added in step (a). In some embodiments, about 5 volumes of solvent are added in step (a). In some embodiments, about 6 volumes of solvent are added in step (a). In some embodiments, about 7 volumes of solvent are added in step (a).

In some embodiments, about 0.50 to about 2.0 equivalents of resolving agent are used in step (a). In some embodiments, about 0.60 to about 1.30 equivalents of resolving agent are used in step (a). In some embodiments, about 0.80 to about 1.30 equivalents of resolving agent are used in step (a). In some embodiments, about 0.95 to about 1.20 equivalents of resolving agent are used in step (a). In some embodiments, about 1.05 equivalents of resolving agent are used in step (a). In some embodiments, about 1.15 equivalents of resolving agent are used in step (a). In various embodiments, about 0.5 to about 2 equivalents, about 0.6 to about 1.5 equivalents, about 0.9 to about 1.5 equivalents, about 1.00 to about 1.10 equivalents, about 0.65 to about 1.25 equivalents, about 0.50 to about 0.60 equivalents, about 0.60 to about 0.70 equivalents, about 0.80 to about 0.90 equivalents, about 0.80 to about 1.30 equivalents, about 0.85 to about 1.25 equivalents, about 0.95 to about 1.05 equivalents, about 0.95 to about 1.20 equivalents, about 0.95 to about 1.20 equivalents, about 1.10 to about 1.20 equivalents, about 1.20 to about 1.30 equivalents, about 0.50 to about 0.60 equivalents, about 0.50 equivalents, about 0.55 equivalents, about 0.60 equivalents, about 0.65 equivalents, about 0.70 equivalents, about 0.75 equivalents, about 0.80 equivalents, about 0.85 equivalents, about 0.90 equivalents, about 0.95 equivalents, about 1.00 equivalents, about 1.05 equivalents, about 1.10 equivalents, about 1.15 equivalents, about 1.20 equivalents, about 1.25 equivalents, or about 1.30 equivalents of resolving agent are used in step (a).

In some embodiments, step (a) comprises: (i) evaporating any solvents present, and (ii) adding diethyl ether to the mixture. In some embodiments, the diethyl ether is removed.

In some embodiments, the mixture in step (a) is seeded with the desired solid salt. For example, when the desired solid salt is (R)-Trolox (1S,2S)-(+)-Pseudoephedrine salt, the mixture may be seeded with (R)-Trolox (1S,2S)-(+)-Pseudoephedrine salt.

In some embodiments, step (b) comprises filtering the solid salt. In some embodiments, step (b) further comprises a step (b)(1), comprising slurrying the solid salt in a solvent. For example, the solid salt filtered from step (b) is added to fresh solvent, and slurried. Step (b)(1) may in some embodiments be used to improve enantiomeric purity. In some embodiments, the slurry solvent is the same solvent used in step (a). In some embodiments, the slurry solvent is a different solvent that the solvent used in step (a). In some embodiments, the slurry time is about 5 minutes to about 5 hours. In some embodiments, the slurry time is about 5 minutes to about 3 hours. In some embodiments, the slurry time is about 5 minutes to about 2 hours. In some embodiments, the slurry time is about 5 minutes to about 1 hour. In some embodiments, the slurry time is about 5 minutes to about 30 minutes. In some embodiments, the slurry time is about 10 minutes to about 20 minutes. In some embodiments, step (b) and/or step (b)(1) further comprise rinsing and drying the solid salt. In some embodiments, the solvent used for rinsing is the same solvent used in step (a). In some embodiments, the slurry used for rinsing is a different solvent that the solvent used in step (a).

In some embodiments, the method further comprises a step (c): (c) separating the Trolox isomer contained in the solid salt from the resolving agent. In some embodiments, step (c) comprises adding an acid to the solid salt. In some embodiments, step (c) comprises adding a base to the solid salt.

In some embodiments, the Trolox isomer that forms the solid salt with the resolving agent is (R)-Trolox. In some embodiments, the Trolox isomer that forms the solid salt with the resolving agent is (S)-Trolox.

In some embodiments, the method comprises: (1) contacting a mixture of (R)-Trolox and (S)-Trolox with about 0.8 to about 1.30 equivalents of (1S,2S)-(+)-Pseudoephedrine and ethyl acetate; (2) heating the mixture until dissolution is achieved; (3) cooling the mixture to about 20° C. to about 30° C. over at least about 50 minutes; (4) cooling the mixture to about 5° C. to about 15° C.; (7) filtering the resulting slurry; (8) washing the wet cake with ethyl acetate; (9) and drying the solids.

In some embodiments, the method comprises: (1) contacting a mixture of (R)-Trolox and (S)-Trolox with about 1.10 to about 1.20 equivalents of (1S,2S)-(+)-Pseudoephedrine and about 4 to about 6 volumes of ethyl acetate; (2) heating the mixture to between about 35° C. to about 55° C. until dissolution is achieved; (3) cooling the mixture to about 20° C. to about 30° C. over at least about 50 minutes; (4) cooling the mixture to about 5° C. to about 15° C. over about 20 to about 40 minutes; (5) holding the temperature in step (4) for about 50-70 minutes; (7) filtering the resulting slurry; (8) washing the wet cake with about 5 to about 7 volumes of ethyl acetate at room temperature; (9) and drying the solids.

In some embodiments, the method comprises: (1) contacting a mixture of (R)-Trolox and (S)-Trolox with about 1.15 equivalents of (1S,2S)-(+)-Pseudoephedrine and about 5 volumes of ethyl acetate; (2) heating the mixture to between about 40° C. to about 50° C. until dissolution is achieved; (3) cooling the mixture to room temperature over at least about 50 minutes; (4) cooling the mixture to about 10° C. over about 30 minutes; (5) holding the temperature in step (4) for about 50-70 minutes; (7) filtering the resulting slurry; (8) washing the wet cake with about 6 volumes of ethyl acetate at room temperature; (9) and drying the solids.

In some embodiments, the enantiomeric excess of the (R)-Trolox obtained from the method is at least about 98%. In some embodiments, the enantiomeric excess of the (R)-Trolox obtained from the method is at least about 99%. In some embodiments, the enantiomeric excess of the (R)-Trolox obtained from the method is at least about 99.5%. In some embodiments, the enantiomeric excess of the (R)-Trolox obtained from the method is at least about 99.9%. In various embodiments, the enantiomeric excess of the (R)-Trolox obtained from the method is at least about 95%, at least about 96%, at least about 97%, at least about 98%, at least about 99%, at least about 99.1%, at least about 99.2%, at least about 99.3%, at least about 99.4% at least about 99.5%, at least about 99.6%, at least about 99.7%, at least about 99.8%, or at least about 99.9%.

In some embodiments, the enantiomeric excess of the (S)-Trolox obtained from the method is at least about 98%. In some embodiments, the enantiomeric excess of the (S)-Trolox obtained from the method is at least about 99%. In some embodiments, the enantiomeric excess of the (S)-Trolox obtained from the method is at least about 99.5%. In some embodiments, the enantiomeric excess of the (S)-Trolox obtained from the method is at least about 99.9%. In various embodiments, the enantiomeric excess of the (S)-Trolox obtained from the method is at least about 95%, at least about 96%, at least about 97%, at least about 98%, at least about 99%, at least about 99.1%, at least about 99.2%, at least about 99.3%, at least about 99.4% at least about 99.5%, at least about 99.6%, at least about 99.7%, at least about 99.8%, or at least about 99.9%.

(R)-Trolox, or a salt thereof, may be produced according to a method described herein. In various embodiments, the enantiomeric excess of the (R)-Trolox or a salt thereof is at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%. (S)-Trolox or a salt thereof, may be produced according to a method described herein. In various embodiments, the enantiomeric excess of the (S)-Trolox or a salt thereof is at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%.

In various embodiments, the purity of the (R)-Trolox or salt thereof, exclusive of any solvents, carriers or excipients, is at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%.

In various embodiments, the purity of the (S)-Trolox or salt thereof, exclusive of any solvents, carriers or excipients, is at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.45% at least about 99.5%, at least about 99.6%, at least about 99.7%, at least about 99.8%, or at least about 99.9%.

The (R)-Trolox that is obtained from the method may be converted to (R)-2-hydroxy-2-methyl-4-(2,4,5-trimethyl-3,6-dioxocyclohexa-1,4-dienyl)butanamide or a salt thereof. The (S)-Trolox that is obtained from the method may be converted to (S)-2-hydroxy-2-methyl-4-(2,4,5-trimethyl-3,6-dioxocyclohexa-1,4-dienyl)butanamide or a salt thereof. In some embodiments, the salt is a pharmaceutically acceptable salt. In some examples, the methods include those steps set forth herein. In some examples, the methods include those in PCT Application No. PCT/US2008/082374.

(R)-2-hydroxy-2-methyl-4-(2,4,5-trimethyl-3,6-dioxocyclohexa-1,4-dienyl)butanamide and (S)-2-hydroxy-2-methyl-4-(2,4,5-trimethyl-3,6-dioxocyclohexa-1,4-dienyl)butanamide may further be converted into the reduced (hydroquinone) forms (i.e. (R)-4-(2,5-dihydroxy-3,4,6-trimethylphenyl)-2-hydroxy-2-methylbutanamide and (S)-4-(2,5-dihydroxy-3,4,6-trimethylphenyl)-2-hydroxy-2-methylbutanamide, respectively). The reduced (hydroxy) form may readily be converted to the oxidized (quinone) form using methods known in the art. See e.g. air, silica Miller et al PCT Intl Appl 2006130775 7 Dec. 2006. The oxidized (quinone) form may readily be converted to the reduced hydroxy form using methods known in the art. See, e.g. Zn, AcOH Fuchs et al EJOC 6 (2009) 833-40. The hydroquinone forms may further be made as salts, in some embodiments, as pharmaceutically acceptable salts.

### Pharmaceutical Compositions

The compounds described herein can be formulated as pharmaceutical compositions (“pharmaceutical compositions” is used interchangeably herein with “pharmaceutical formulations”) by formulation with additives such as pharmaceutically acceptable excipients, pharmaceutically acceptable carriers, and pharmaceutically acceptable vehicles. “Pharmaceutically acceptable excipients”, “pharmaceutically acceptable carriers”, and “pharmaceutically acceptable vehicles” are used interchangeably herein. Suitable pharmaceutically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21st edition (2005), incorporated herein by reference.

A pharmaceutical composition can comprise a unit dose formulation, where the unit dose is a dose sufficient to have a therapeutic or suppressive effect. The unit dose may be sufficient as a single dose to have a therapeutic or suppressive effect. Alternatively, the unit dose may be a dose administered periodically in a course of treatment or suppression of a disorder.

Pharmaceutical compositions containing the compounds of the invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Time-release or controlled release delivery systems may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The compounds of the invention may be administered enterally, orally, parenterally, sublingually, by inhalation (e.g. as mists or sprays), rectally, or topically in dosage unit formulations containing conventional nontoxic pharmaceutically acceptable carriers, adjuvants, and vehicles as desired. For example, suitable modes of administration include oral, subcutaneous, transdermal, transmucosal, iontophoretic, intravenous, intraarterial, intramuscular, intraperitoneal, intranasal (e.g. via nasal mucosa), subdural, rectal, gastrointestinal, and the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous, intramuscular, intrasternal injection, or infusion techniques. The compounds are mixed with pharmaceutically acceptable carriers, adjuvants, and vehicles appropriate for the desired route of administration. Oral administration is a preferred route of administration, and formulations suitable for oral administration are preferred formulations. The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. Additional methods of administration are known in the art.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

The invention also provides articles of manufacture and kits containing materials useful for treating or suppressing oxidative stress disorder. In some embodiments, the kit of the invention comprises the container described above.

In other aspects, the kits may be used for any of the methods described herein, including, for example, to treat an individual with an oxidative stress disorder, or to suppress an oxidative stress disorder in an individual.

Diseases Amenable to Treatment or Suppression with Compounds, Compositions and Methods of the Invention

A variety of disorders/diseases are believed to be caused or aggravated by oxidative stress affecting normal electron flow in the cells, such as mitochondrial disorders, impaired energy processing disorders, neurodegenerative diseases and diseases of aging, and can be treated or suppressed using compounds and methods of the invention.

Non-limiting examples of oxidative stress disorders include, for example, mitochondrial disorders (including inherited mitochondrial diseases) such as Alpers Disease, Barth syndrome, Beta-oxidation Defects, Carnitine-Acyl-Carnitine Deficiency, Carnitine Deficiency, Creatine Deficiency Syndromes, Co-Enzyme Q10 Deficiency, Complex I Deficiency, Complex II Deficiency, Complex III Deficiency, Complex IV Deficiency, Complex V Deficiency, COX Deficiency, chronic progressive external ophthalmoplegia (CPEO), CPT I Deficiency, CPT II Deficiency, Friedreich's Ataxia (FA), Glutaric Aciduria Type II, Kearns-Sayre Syndrome (KSS), Lactic Acidosis, Long-Chain Acyl-CoA Dehydrongenase Deficiency (LCAD), LCHAD, Leigh Disease or Syndrome, Leigh-like Syndrome, Leber's Hereditary Optic Neuropathy (LHON, also referred to as Leber's Disease, Leber's Optic Atrophy (LOA), or Leber's Optic Neuropathy (LON)), Lethal Infantile Cardiomyopathy (LIC), Luft Disease, Multiple Acyl-CoA Dehydrogenase Deficiency (MAD), Medium-Chain Acyl-CoA Dehydrongenase Deficiency (MCAD), Mitochondrial Myopathy, Encephalopathy, Lactacidosis, Stroke (MELAS), Myoclonic Epilepsy with Ragged Red Fibers (MERRF), Mitochondrial Recessive Ataxia Syndrome (MIRAS), Mitochondrial Cytopathy, Mitochondrial DNA Depletion, Mitochondrial Encephalopathy, Mitochondrial Myopathy, Myoneurogastrointestinal Disorder and Encephalopathy (MNGIE), Neuropathy, Ataxia, and Retinitis Pigmentosa (NARP), Pearson Syndrome, Pyruvate Carboxylase Deficiency, Pyruvate Dehydrogenase Deficiency, POLG Mutations, Respiratory Chain Disorder, Short-Chain Acyl-CoA Dehydrogenase Deficiency (SCAD), SCHAD, Very Long-Chain Acyl-CoA Dehydrongenase Deficiency (VLCAD); myopathies such as cardiomyopathy and encephalomyopathy; neurodegenerative diseases such as Parkinson's disease, Alzheimer's disease, and amyotrophic lateral sclerosis (ALS, also known as Lou Gehrig's disease); motor neuron diseases; neurological diseases such as epilepsy; age-associated diseases, particularly diseases for which CoQ10 has been proposed for treatment, such as macular degeneration, diabetes (e.g. Type 2 diabetes mellitus), metabolic syndrome, and cancer (e.g. brain cancer); genetic diseases such as Huntington's Disease (which is also a neurological disease); mood disorders such as schizophrenia and bipolar disorder; pervasive developmental disorders such as autistic disorder, Asperger's syndrome, childhood disintegrative disorder (CDD), Rett's disorder, and PDD-not otherwise specified (PDD-NOS); cerebrovascular accidents such as stroke; vision impairments such as those caused by neurodegenerative diseases of the eye such as optic neuropathy, Leber's hereditary optic neuropathy, dominant inherited juvenile optic atrophy, optic neuropathy caused by toxic agents, glaucoma, age-related macular degeneration (both “dry” or non-exudative macular degeneration and “wet” or exudative macular degeneration), Stargardt's macular dystrophy, diabetic retinopathy, diabetic maculopathy, retinopathy of prematurity, or ischemic reperfusion-related retinal injury; disorders caused by energy impairment include diseases due to deprivation, poisoning or toxicity of oxygen, and qualitative or quantitative disruption in the transport of oxygen such as haemoglobionopathies, for example thalassemia or sickle cell anemia; other diseases in which mitochondrial dysfunction is implicated such as excitoxic, neuronal injury, such as that associated with seizures, stroke and ischemia; and other disorders including renal tubular acidosis; attention deficit/hyperactivity disorder (ADHD); neurodegenerative disorders resulting in hearing or balance impairment; Dominant Optic Atrophy (DOA); Maternally inherited diabetes and deafness (MIDD); chronic fatigue; contrast-induced kidney damage; contrast-induced retinopathy damage; Abetalipoproteinemia; retinitis pigmentosum; Wolfram's disease; Tourette syndrome; cobalamin c defect; methylmalonic aciduria; glioblastoma; Down's syndrome; acute tubular necrosis; muscular dystrophies; leukodystrophies; Progressive Supranuclear Palsy; spinal muscular atrophy; hearing loss (e.g. noise induced hearing loss); traumatic brain injury; Juvenile Huntington's Disease; Multiple Sclerosis; NGLY1; Multiple System Atrophy; Adrenoleukodystrophy; and Adrenomyeloneuropathy. It is to be understood that certain specific diseases or disorders may fall within more than one category; for example, Huntington's Disease is a genetic disease as well as a neurological disease. Furthermore, certain oxidative stress diseases and disorders may also be considered mitochondrial disorders.

For some disorders amenable to treatment with compounds and methods of the invention, the primary cause of the disorder is due to a defect in the respiratory chain or another defect preventing normal utilization of energy in mitochondria, cells, or tissue(s). Non-limiting examples of disorders falling in this category include inherited mitochondrial diseases, such as Myoclonic Epilepsy with Ragged Red Fibers (MERRF), Mitochondrial Myopathy, Encephalopathy, Lactacidosis, and Stroke (MELAS), Leber's Hereditary Optic Neuropathy (LHON, also referred to as Leber's Disease, Leber's Optic Atrophy (LOA), or Leber's Optic Neuropathy (LON)), Leigh Disease or Leigh Syndrome, Kearns-Sayre Syndrome (KSS), and Friedreich's Ataxia (FA). For some disorders amenable to treatment with compounds and methods of the invention, the primary cause of the disorder is not due to respiratory chain defects or other defects preventing normal utilization of energy in mitochondria, cells, or tissue(s); non-limiting examples of disorders falling in this category include stroke, cancer, and diabetes. However, these latter disorders are particularly aggravated by energy impairments, and are particularly amenable to treatment with compounds of the invention in order to ameliorate the condition. Pertinent examples of such disorders include ischemic stroke and hemorrhagic stroke, where the primary cause of the disorder is due to impaired blood supply to the brain. While an ischemic episode caused by a thrombosis or embolism, or a hemorrhagic episode caused by a ruptured blood vessel, is not primarily caused by a defect in the respiratory chain or another metabolic defect preventing normal utilization of energy, oxidative stress plays a role in the ischemic cascade due to oxygen reperfusion injury following hypoxia (this cascade occurs in heart attacks as well as in strokes). Accordingly, treatment with compounds and methods of the invention will mitigate the effects of the disease, disorder or condition.

The term “oxidative stress disorder” or “oxidative stress disease” encompass both diseases caused by oxidative stress and diseases aggravated by oxidative stress. The terms “oxidative stress disorder” or “oxidative stress disease” encompass both diseases and disorders where the primary cause of the disease is due to a defect in the respiratory chain or another defect preventing normal utilization of energy in mitochondria, cells, or tissue(s), and also diseases and disorders where the primary cause of the disease is not due to a defect in the respiratory chain or another defect preventing normal utilization of energy in mitochondria, cells, or tissue(s). The former set of diseases can be referred to as “primary oxidative stress disorders,” while the latter can be referred to as “secondary oxidative stress disorders.” It should be noted that the distinction between “diseases caused by oxidative stress” and “diseases aggravated by oxidative stress” is not absolute; a disease may be both a disease caused by oxidative stress and a disease aggravated by oxidative stress. The boundary between “primary oxidative stress disorder” and a “secondary oxidative stress disorder” is more distinct, provided that there is only one primary cause of a disease or disorder and that primary cause is known.

Bearing in mind the somewhat fluid boundary between diseases caused by oxidative stress and diseases aggravated by oxidative stress, mitochondrial diseases or disorders and impaired energy processing diseases and disorders tend to fall into the category of diseases caused by oxidative stress, while neurodegenerative disorders and diseases of aging tend to fall into the category of diseases aggravated by oxidative stress. Mitochondrial diseases or disorders and impaired energy processing diseases and disorders are generally primary oxidative stress disorders, while neurodegenerative disorders and diseases of aging may be primary or secondary oxidative stress disorders.

The compounds of the invention may be further be used in treatment or prophylactic treatment against radiation exposure. Further description of the disorders that can be treated with the compounds of the invention is found in U.S. Provisional Patent Application No. 62/092,743; and PCT Application Nos. PCT/US08/082374; PCT/US09/035996; and PCT/US09/060489.

Synthetic Reaction Parameters

Solvents employed in synthesis of the compounds and compositions of the invention include, for example, water, acetonitrile (“ACN”), diethyl ether, 2-methyl-tetrahydrofuran (“2-MeTHF”), ethyl acetate (“EtOAc”), ethanol (“EtOH”), isopropyl alcohol (“IPA”), isopropyl acetate (“IPAc”), methanol (MeOH), and the like, as well as mixtures thereof.

The term “q.s.” means adding a quantity sufficient to achieve a stated function, e.g., to bring a solution to the desired volume (i.e., 100%).

Techniques useful in synthesizing the compounds and compositions herein are both readily apparent and accessible to those of skill in the relevant art in light of the teachings described herein. The discussion below is offered to illustrate certain of the diverse methods available for use in assembling the compounds and compositions herein. However, the discussion is not intended to define the scope of reactions or reaction sequences that are useful in preparing the compounds and compositions herein.

Other methods for producing the compounds and compositions of the invention will be apparent to one skilled in the art in view of the teachings herein.

### EXAMPLES

### Example 1

### Solubility Assessment of (R)-Trolox

(R)-Trolox (97.89% ee, chemical purity>99.99% AUC) (100 mg) was weighed out in 12 different vials. 12 different solvents were added (one volume at a time) to the different vials and the mixture was maintained at 40° C. The volume of each solvent required to dissolve 100 mg of (R)-Trolox was recorded and the solubility was subsequently calculated. Solubility data is presented in Table 1.

TABLE 1
Solubility assessment of (R)-Trolox

| | | Amt of solvent | Solubility |
|---|---|---|---|
| | | at 40° C. | at 40° C. |
| (R)-Trolox (mg) | Solvent | (μL) | (mg/mL) |
| 97.7 | water | >3.5 mL | not soluble |
| 100.4 | MeOH | 100 | 1004 |
| 98.7 | EtOH | 200 | 494 |
| 104.5 | IPA | 200 | 523 |
| 100.8 | IPAc | 400 | 252 |
| 104.6 | EtOAc | 500 | 209 |
| 97.4 | ACN | 500 | 195 |
| 104.8 | 2-MeTHF | 200 | 524 |
| 103.9 | 5% water/MeOH | 100 | 1039 |
| 102.5 | 5% water/EtOH | 120 | 854 |
| 97.8 | 5% water/IPA | 100 | 978 |
| 100.9 | 5% water/ACN | 220 | 459 |

(R)-Trolox was found to be highly soluble in all solvents except for pure water. Isopropyl acetate (IPAc), 2-methyltetrahydrofuran (2-MeTHF), and 5% water/isopropyl alcohol (v/v) were chosen for the initial salt screening experiments.

### Example 2

### Initial Salt Screening Experiments Using (R)-Trolox

(R)-Trolox (4.00 g) was weighed out in a vial and dissolved in ˜20 mL of EtOH. The total volume of this solution was measured out to be 22.3 mL. Therefore, 0.558 mL of this solution corresponded to 100 mg of (R)-Trolox. 1.05 eq. (100 mg of (R)-Trolox=1 eq.) of each base was weighed out in 39 different vials (3 vials per base). 0.558 mL of the (R)-Trolox solution was added to each vial and the solvent was evaporated slowly in a vacuum oven at RT. 0.500 mL of each solvent (a: IPAc, b: 2-MeTHF, and c: 5% water/IPA) was added to the appropriate vial and the solution was stirred at 40° C. for 1.5 hours using magnetic stirrer. Then, the solution was cooled down to RT for at least 1.5 hours. If solids precipitated, they were filtered and analyzed by XRPD and optical microscopy. If no solids precipitated, the solvent was evaporated slowly (uncapping of the vial) at RT overnight. If solids were obtained after evaporation, they were analyzed by XRPD and optical microscopy. If no solids were obtained after evaporation, 0.500 mL of diethyl ether was added to gel in the vial and stirred. If solids precipitated, they were filtered and analyzed by XRPD and optical microscopy. If no solids precipitated, the solvent was evaporated slowly (uncapping of the vial) at RT overnight.

TABLE 2
Reagents used for the initial salt screening experiments

| Reagent | | MW | density | mmol | equi | amount | Supplier |
|---|---|---|---|---|---|---|---|
| R-trolox | | 250.29 | n/a | 0.400 | 1 | 100.0 | JMPS |
| 1 | (R)-(+)-N-Benzyl- | 211.30 | 1.01 | 0.420 | 1.05 | 87.8 | Aldrich |
| | α-methylbenzylamine | | | | | | |
| 2 | (1S,2S)-(+)-N- | 179.26 | n/a | 0.420 | 1.05 | 75.2 | Aldrich |
| | methylpseudoephedrine | | | | | | |
| 3 | (R)-(−)-Epinephrine | 183.20 | n/a | 0.420 | 1.05 | 76.9 | Sigma |
| 4 | (R)-(+)-2-Amino-3- | 151.21 | n/a | 0.420 | 1.05 | 63.4 | Aldrich |
| | phenyl-1-propanol | | | | | | |
| 5 | (1S,2S)-(+)- | 213.30 | n/a | 0.420 | 1.05 | 89.5 | Aldrich |
| | Thiomicamine | | | | | | |
| 6 | (+)-Cinchonine | 294.39 | n/a | 0.420 | 1.05 | 123.5 | Aldrich |
| 7 | N-methyl-D-glucamine | 195.21 | n/a | 0.420 | 1.05 | 81.9 | Alfa |
| | | | | | | | Aesar |
| 8 | (R)-(+)-1-(1-Naphthyl)- | 171.24 | 1.067 | 0.420 | 1.05 | 67.3 | Fluka |
| | ethylamine | | | | | | |
| 9 | (1S,2S)-(+)- | 165.23 | n/a | 0.420 | 1.05 | 69.3 | Aldrich |
| | Pseudoephedrine | | | | | | |
| 10 | D-Lysine | 146.19 | n/a | 0.420 | 1.05 | 61.3 | Sigma |
| 11 | L-Lysine | 146.19 | n/a | 0.420 | 1.05 | 61.3 | Aldrich |
| 12 | L-Histidine | 155.15 | n/a | 0.420 | 1.05 | 65.1 | Alfa |
| | | | | | | | Aesar |
| 13 | L-Arginine | 174.20 | n/a | 0.420 | 1.05 | 73.1 | Alfa |
| | | | | | | | Aesar |

In the following Tables 3-4, (a) refers to Isopropyl acetate (IPAc), (b) refers to 2-methyltetrahydrofuran (2-MeTHF), and (c) refers to 5% water/isopropyl alcohol (v/v). The base numbers in Tables 3 and 4 correspond to the bases numbered 1-13 in Table 2.

TABLE 3
Results from the initial salt screening experiments using (R)-Trolox

| | | | | | | After |
|---|---|---|---|---|---|---|
| | | | After | | After | evaporation |
| | | | evaporation | In solvent | cooling | from solvent |
| Exp | Base | In EtOH | from EtOH | a/b/c at 40° C. | down | a/b/c |
| 1a | 1 | yellow | yellow gel | yellow | yellow | yellow gel |
| | | solution | | solution | solution | |
| 1b | | yellow | yellow gel | yellow | yellow | yellow gel |
| | | solution | | solution | solution | |
| 1c | | yellow | yellow gel | yellow | yellow | yellow gel |
| | | solution | | solution | solution | |
| 2a | 2 | yellow | yellow gel | white solid | white solid | N/A |
| | | solution | | slurry | slurry | |
| 2b | | yellow | yellow gel | yellow | yellow | yellow gel |
| | | solution | | solution | solution | |
| 2c | | yellow | yellow gel | yellow | yellow | yellow gel |
| | | solution | | solution | solution | |
| 3a | 3 | base did not | thick slurry | thick off | thick off | N/A |
| | | dissolve | | white slurry | white slurry | |
| 3b | | base did not | thick slurry | off white | off white | N/A |
| | | dissolve | | slurry | slurry | |
| 3c | | base did not | thick slurry | off white | off white | N/A |
| | | dissolve | | slurry | slurry | |
| 4a | 4 | bright yellow | bright | white solids | white solids, | N/A |
| | | solution | yellow gel | | add | |
| | | | | | 0.500 mL | |
| | | | | | solvent | |
| 4b | | bright yellow | bright | bright yellow | bright | yellow waxy |
| | | solution | yellow gel | solution | yellow | solid |
| | | | | | solution | |
| 4c | | bright yellow | bright | bright yellow | bright | yellow waxy |
| | | solution | yellow gel | solution | yellow | solid |
| | | | | | solution | |
| 5a | 5 | solid? | thick slurry | thin yellow | thin yellow | N/A |
| | | | | slurry | slurry | |
| 5b | | solid? | thick slurry | yellow | thin yellow | N/A |
| | | | | solution | slurry | |
| 5c | | solid? | thick slurry | yellow | yellow | some yellow |
| | | | | solution | solution | solid |
| 6a | 6 | base did not | thick slurry | off white | off white | N/A |
| | | dissolve | | slurry | slurry | |
| 6b | | base did not | thick slurry | off white | off white | N/A |
| | | dissolve | | slurry | slurry | |
| 6c | | base did not | thick slurry | off white | off white | N/A |
| | | dissolve | | slurry | slurry | |
| 7a | 7 | base did not | thick slurry | white solid | forced to stir | N/A |
| | | dissolve | | chunks, clear | | |
| | | | | liquid, not | | |
| | | | | stirring | | |
| 7b | | base did not | thick slurry | white solids | white solids, | N/A |
| | | dissolve | | | add | |
| | | | | | 0.500 mL | |
| | | | | | solvent | |
| 7c | | base did not | thick slurry | white solids | white solids, | N/A |
| | | dissolve | | | add | |
| | | | | | 0.500 mL | |
| | | | | | solvent | |
| 8a | 8 | orange | orange gel | orange | orange | orange gel |
| | | solution | | solution | solution | |
| 8b | | orange | orange gel | orange | orange | orange gel |
| | | solution | | solution | solution | |
| 8c | | orange | orange gel | orange | orange | orange gel |
| | | solution | | solution | solution | |
| 9a | 9 | yellow | yellow gel | yellow | yellow | yellow gel |
| | | solution | | solution | solution | |
| 9b | | yellow | yellow gel | yellow | yellow | yellow gel |
| | | solution | | solution | solution | |
| 9c | | yellow | yellow gel | yellow | yellow | yellow gel |
| | | solution | | solution | solution | |
| 10a | 10 | base did not | thick slurry | white solid | white solid | N/A |
| | | dissolve | | chunks, | chunks, | |
| | | | | clear liquid | clear liquid | |
| 10b | | base did not | thick slurry | white solid | waxy, not | N/A |
| | | dissolve | | chunks, | stirring | |
| | | | | clear liquid | | |
| 10c | | base did not | thick slurry | off white | | N/A |
| | | dissolve | | slurry | | |
| 11a | 11 | base did not | thick slurry | white solid | white solid | N/A |
| | | dissolve | | chunks, | chunks, | |
| | | | | clear liquid | clear liquid | |
| 11b | | base did not | thick slurry | white solid | white solid | N/A |
| | | dissolve | | chunks, | chunks, | |
| | | | | clear liquid | clear liquid | |
| 11c | | base did not | thick slurry | thin yellow | waxy solid | N/A |
| | | dissolve | | slurry | slurry | |
| 12a | 12 | base did not | thick slurry | white slurry | white slurry | N/A |
| | | dissolve | | | | |
| 12b | | base did not | thick slurry | white slurry | white slurry | N/A |
| | | dissolve | | | | |
| 12c | | base did not | thick slurry | white slurry | white slurry | N/A |
| | | dissolve | | | | |
| 13a | 13 | base did not | thick slurry | white solid | white solid | N/A |
| | | dissolve | | chunks, | chunks, | |
| | | | | clear liquid | clear liquid | |
| 13b | | base did not | thick slurry | white solid | white solid | N/A |
| | | dissolve | | chunks, | chunks, | |
| | | | | clear liquid | clear liquid | |
| 13c | | base did not | thick slurry | thin yellow | waxy solid | N/A |
| | | dissolve | | slurry | slurry | |

TABLE 4
Results from the initial salt screening experiments using (R)-Trolox
(continued)

| | | After | After | | |
|---|---|---|---|---|---|
| | | addition | evaporation | | |
| | | of diethyl | from diethyl | | |
| Exp | Base | ether | ether | XRPD | On Plate |
| 1a | 1 | yellow | yellow gel | no solid | N/A |
| | | solution | | | |
| 1b | | yellow | yellow gel | no solid | N/A |
| | | solution | | | |
| 1c | | yellow | yellow gel | no solid | N/A |
| | | solution | | | |
| 2a | 2 | N/A | N/A | crystalline salt | white solid |
| 2b | | waxy solid | N/A | unable to filter | N/A |
| | | slurry | | | |
| 2c | | waxy solid | N/A | unable to filter | N/A |
| | | slurry | | | |
| 3a | 3 | N/A | N/A | base/salt | off white solid |
| | | | | mixture | |
| 3b | | N/A | N/A | base | off white solid |
| 3c | | N/A | N/A | base/salt | off white solid |
| | | | | mixture | |
| 4a | 4 | N/A | N/A | crystalline salt | white solid |
| 4b | | N/A | N/A | crystalline salt | yellow waxy |
| | | | | | solid |
| 4c | | N/A | N/A | crystalline salt | yellow waxy |
| | | | | | solid |
| 5a | 5 | N/A | N/A | base | white solid |
| 5b | | N/A | N/A | base | white solid |
| 5c | | waxy solid | N/A | unable to filter | N/A |
| | | slurry | | | |
| 6a | 6 | N/A | N/A | base | off white solid |
| 6b | | N/A | N/A | base | off white solid |
| 6c | | N/A | N/A | base | off white solid |
| 7a | 7 | N/A | N/A | crystalline salt | white solid |
| 7b | | N/A | N/A | crystalline salt | white solid |
| 7c | | N/A | N/A | crystalline salt | white solid |
| 8a | 8 | orange | orange gel | no solid | N/A |
| | | solution | | | |
| 8b | | orange | orange gel | no solid | N/A |
| | | solution | | | |
| 8c | | orange | orange gel | no solid | N/A |
| | | solution | | | |
| 9a | 9 | white | N/A | crystalline salt | white solid |
| | | solid | | | |
| | | slurry | | | |
| 9b | | waxy solid | N/A | unable to filter | N/A |
| | | slurry | | | |
| 9c | | yellow | yellow gel | no solid | N/A |
| | | solution | | | |
| 10a | 10 | N/A | N/A | amorphous | waxy solid |
| 10b | | N/A | N/A | amorphous | waxy solid |
| 10c | | N/A | N/A | crystalline salt | white solid |
| 11a | 11 | N/A | N/A | amorphous | waxy solid |
| 11b | | N/A | N/A | amorphous | white solid |
| 11c | | N/A | N/A | unable to filter | N/A |
| 12a | 12 | N/A | N/A | base | white solid |
| 12b | | N/A | N/A | base | white solid |
| 12c | | N/A | N/A | base | white solid |
| 13a | 13 | N/A | N/A | amorphous | white solid |
| 13b | | N/A | N/A | amorphous | white solid |
| 13c | | N/A | N/A | unable to filter | N/A |

After initial XRPD screening, 6 crystalline salts were isolated and further analyzed by ¹H-NMR and optical microscopy. The bases that produced salts with (R)-Trolox were (1S,2S)-(+)-N-methylpseudoephedrine, (R)-(−)-Epinephrine, (R)-(+)-2-Amino-3-phenyl-1-propanol, N-methyl-D-glucamine, (1S,2S)-(+)-Pseudoephedrine and D-Lysine.

### Example 3

### Additional Salt Screening Experiments Using (R)-Trolox

(R)-Trolox (1.70 g) was weighed out in a vial and dissolved in ˜8.5 mL of EtOH. The total volume of this solution was measured out to be 8.8 mL. Therefore, 0.518 mL of this solution corresponded to 100 mg of (R)-Trolox. 1.05 eq. (100 mg of (R)-Trolox=1 eq.) of each base was weighed out in 12 different vials (3 vials per base). 0.518 mL of the (R)-Trolox solution was added to each vial (plus 3 empty vials as controls) and the solvent was evaporated slowly in a vacuum oven at RT. 0.500 mL of each solvent (a: IPAc, b: 2-MeTHF, and c: 5 water/IPA) was added to the appropriate vial and the solution was stirred at 40° C. for 1.5 hours using magnetic stirrer. Then, the solution was cooled down to RT for at least 1.5 hours. If solids precipitated, they were filtered and analyzed by XRPD and optical microscopy. If no solids precipitated, the solvent was evaporated slowly (uncapping of the vial) at RT overnight. If solids were obtained after evaporation, they were analyzed by XRPD and optical microscopy. If no solids were obtained after evaporation, 0.500 mL of diethyl ether was added to gel in the vial and stirred. If solids precipitated, they were filtered and analyzed by XRPD and optical microscopy. If no solids precipitated, the solvent was evaporated slowly (uncapping of the vial) at RT overnight.

TABLE 5
Reagents used for the additional salt screening experiments using (R)-Trolox

| | Reagent | MW | density | mmol | equi | amount | Supplier |
|---|---|---|---|---|---|---|---|
| | R-trolox | 250.29 | n/a | 0.400 | 1 | 100.0 | JMPS |
| 14. | Dehydroabietylamine | 285.47 | n/a | 0.420 | 1.05 | 119.8 | Aldrich |
| 15. | (R)-(−)-Leucinol | 117.19 | 0.9 | 0.420 | 1.05 | 54.6 | Alfa |
| | | | | | | | Aesar |
| 16. | (R)-(−)-2-Amino-3- | 103.16 | n/a | 0.420 | 1.05 | 43.3 | Alfa |
| | methyl-1-butanol | | | | | | Aesar |
| 17. | (R)-(−)-2-Amino-1- | 75.11 | 0.963 | 0.420 | 1.05 | 32.7 | Alfa |
| | propanol | | | | | | Aesar |

In the following Tables 6 and 7, (a) refers to Isopropyl acetate (IPAc), (b) refers to 2-methyltetrahydrofuran (2-MeTHF), and (c) refers to 5% water/isopropyl alcohol (v/v). The base numbers in Tables 6 and 7 correspond to the bases numbered 14-17 in Table 5.

TABLE 6
Results from the additional salt screening experiments using (R)-Trolox

| | | | | | | After |
|---|---|---|---|---|---|---|
| | | | After | | After | evaporation |
| | | | evaporation | In solvent | cooling | from solvent |
| Exp | Base | In EtOH | from EtOH | a/b/c at 40° C. | down | a/b/c |
| 14a | 14 | yellow | yellow gel | yellow solution | yellow | yellow gel |
| | | solution | | | solution | |
| 14b | | yellow | yellow gel | yellow solution | yellow | yellow gel |
| | | solution | | | solution | |
| 14c | | yellow | yellow gel | yellow solution | yellow | yellow gel |
| | | solution | | | solution | |
| 15a | 15 | yellow | yellow gel | yellow solution | yellow | yellow gel |
| | | solution | | | solution | |
| 15b | | yellow | yellow gel | yellow solution | yellow | yellow gel |
| | | solution | | | solution | |
| 15c | | yellow | yellow gel | yellow solution | yellow | yellow gel |
| | | solution | | | solution | |
| 16a | 16 | yellow | yellow gel | yellow solution | yellow | yellow gel |
| | | solution | | | solution | |
| 16b | | yellow | yellow gel | yellow solution | yellow | yellow gel |
| | | solution | | | solution | |
| 16c | | yellow | yellow gel | yellow solution | yellow | yellow gel |
| | | solution | | | solution | |
| 17a | 17 | yellow | yellow gel | yellow solution | cloudy | oily bottom |
| | | solution | | | yellow | layer, cloudy |
| | | | | | solution | top layer |
| 17b | | yellow | yellow gel | yellow solution | yellow | yellow gel |
| | | solution | | | solution | |
| 17c | | yellow | yellow gel | yellow solution | yellow | yellow gel |
| | | solution | | | solution | |
| a | no | yellow | white solid | yellow solution | yellow | white solid |
| | base | solution | | | solution | |
| b | | yellow | white solid | yellow solution | yellow | white solid |
| | | solution | | | solution | |
| c | | yellow | white solid | yellow solution | yellow | white solid |
| | | solution | | | solution | |

TABLE 7
Results from the additional salt screening experiments using (R)-Trolox
(continued)

| | | | After |
|---|---|---|---|
| | | After | evaporation |
| | | addition of | from diethyl |
| Exp | Base | diethyl ether | ether |
| 14a | 14 | solid gel | N/A |
| 14b | | yellow | yellow gel |
| | | solution | |
| 14c | | yellow | yellow gel |
| | | solution | |
| 15a | 15 | oily bottom | N/A |
| | | layer, clear | |
| | | top layer | |
| 15b | | oily bottom | N/A |
| | | layer, clear | |
| | | top layer | |
| 15c | | yellow | yellow gel |
| | | solution | |
| 16a | 16 | oily bottom | N/A |
| | | layer, clear | |
| | | top layer | |
| 16b | | oily bottom | N/A |
| | | layer, clear | |
| | | top layer | |
| 16c | | cloudy | yellow gel |
| | | yellow | |
| | | solution | |
| 17a | 17 | N/A | N/A |
| 17b | | oily bottom | N/A |
| | | layer, clear | |
| | | top layer | |
| 17c | | oily bottom | N/A |
| | | layer, clear | |
| | | top layer | |
| a | no | thick white | N/A |
| | base | slurry | |
| b | | thick white | N/A |
| | | slurry | |
| c | | thick white | N/A |
| | | slurry | |

No crystalline salts were isolated.

### Example 4

### Salt Screening Experiments Using (S)-Trolox and the Bases that Produced Salts with (R)-Trolox

1.05 eq. (100 mg of (S)-Trolox=1 eq.) of each base was weighed out in 6 different vials. (S)-Trolox (100 mg) was added to each vial. 0.500 mL of the appropriate solvent (IPAc or 5% water/IPA) was added to the appropriate vial and the solution was stirred at 40° C. for 1.5 hours using magnetic stirrer. Then, the solution was cooled down to RT for at least 1.5 hours. If solids precipitated, they were filtered and analyzed by XRPD and optical microscopy. If no solids precipitated, the solvent was evaporated slowly (uncapping of the vial) at RT overnight. If solids were obtained after evaporation, they were analyzed by XRPD and optical microscopy. If no solids were obtained after evaporation, 0.500 mL of diethyl ether was added to gel in the vial and stirred. If solids precipitated, they were filtered and analyzed by XRPD and optical microscopy. If no solids precipitated, the solvent was evaporated slowly (uncapping of the vial) at RT overnight.

TABLE 8
Reagents used for the salt screening experiments using (S)-
Trolox and the bases that produced salts with (R)-Trolox

| Reagent | | MW | Density | mmol | equi | amount | Supplier | Solvent |
|---|---|---|---|---|---|---|---|---|
| S-trolox | | 250.29 | n/a | 0.400 | 1 | 100.0 | JMPS | N/A |
| 2 | (1S,2S)-(+)-N- | 179.26 | n/a | 0.420 | 1.05 | 75.2 | Aldrich | IPAc |
| | methylpseudo- | | | | | | | |
| | ephedrine | | | | | | | |
| 3 | (R)-(−)- | 183.20 | n/a | 0.420 | 1.05 | 76.9 | Sigma | 5% |
| | Epinephrine | | | | | | | water/IPA |
| 4 | (R)-(+)-2-Amino-3- | 151.21 | n/a | 0.420 | 1.05 | 63.4 | Aldrich | IPAc |
| | phenyl-1-propanol | | | | | | | |
| 7 | N-methyl-D- | 195.21 | n/a | 0.420 | 1.05 | 81.9 | Alfa | 5% |
| | glucamine | | | | | | Aesar | water/IPA |
| 9 | (1S,2S)-(+)- | 165.23 | n/a | 0.420 | 1.05 | 69.3 | Aldrich | IPAc |
| | Pseudoephedrine | | | | | | | |
| 10 | D-Lysine | 146.19 | n/a | 0.420 | 1.05 | 61.3 | Sigma | 5% |
| | | | | | | | | water/IPA |

TABLE 9
Results from the salt screening experiments using (S)-Trolox
and the bases that produced salts with (R)-Trolox

| | | | | After | After | After |
|---|---|---|---|---|---|---|
| | | In solvent | In solvent | cooling | evaporation | addition of |
| Exp | Base | at RT | at 40° C. | down | from solvent | diethyl ether |
| 2 | 2 | white solid | white solid | frozen | N/A | N/A |
| | | | | white/yellow | | |
| | | | | solid | | |
| 3 | 3 | off white | off white | off white | N/A | N/A |
| | | slurry | slurry | slurry | | |
| 4 | 4 | bright yellow | bright yellow | bright yellow | bright | yellow oily |
| | | solution | solution | solution | yellow gel | bottom layer, |
| | | | | | | cloudy top |
| | | | | | | layer |
| 7 | 7 | cloudy yellow | yellow | thin yellow | N/A | N/A |
| | | solution | solution | slurry | | |
| 9 | 9 | yellow | yellow | yellow | yellow gel | oily bottom |
| | | solution | solution | solution | | layer, cloudy |
| | | | | | | top layer |
| 10 | 10 | cloudy yellow | yellow slurry | thin yellow | N/A | N/A |
| | | solution with | | slurry | | |
| | | white solid | | | | |

TABLE 10
Results from the salt screening experiments using (S)-Trolox and
the bases that produced salts with (R)-Trolox (continued)

| | | | | Comparison to R- |
|---|---|---|---|---|
| Exp | Base | XRPD | On Plate | Trolox salt XRPD |
| 2 | 2 | crystalline salt | white solid | different |
| | | | | crystalline salt |
| 3 | 3 | mixture of | white solid | mixture of |
| | | crystalline | | crystalline salt and |
| | | salt and base | | base |
| 4 | 4 | no solid | N/A | N/A |
| 7 | 7 | base | waxy solid | crystalline salt |
| 9 | 9 | no solid | N/A | N/A |
| 10 | 10 | unable to filter | N/A | N/A |

The promising bases that formed solid salts with (R)-Trolox but very little or no solid with (S)-Trolox were (R)-(+)-2-Amino-3-phenyl-1-propanol, N-methyl-D-glucamine, (1S,2S)-(+)-Pseudoephedrine and D-Lysine.

### Example 5

### Repeat of Experiments Using (R)-Trolox and the 4 Bases that Previously Produced Salts with (R)-Trolox but Did Not Produce Salts with (S)-Trolox

1.05 eq. (100 mg of (R)-Trolox=1 eq.) of each base was weighed out in 4 different vials. (R)-Trolox (100 mg) was added to each vial. 0.500 mL of the appropriate solvent (IPAc or 5% water/IPA) was added to the appropriate vial and the solution was stirred at 40° C. for 1.5 hours using magnetic stirrer. Then, the solution was cooled down to RT for at least 1.5 hours. If solids precipitated, they were filtered and analyzed by XRPD and optical microscopy. If no solids precipitated, the solvent was evaporated slowly (uncapping of the vial) at RT overnight. If solids were obtained after evaporation, they were analyzed by XRPD and optical microscopy. If no solids were obtained after evaporation, 0.500 mL of diethyl ether was added to gel in the vial and stirred. If solids precipitated, they were filtered and analyzed by XRPD and optical microscopy. If no solids precipitated, the solvent was evaporated slowly (uncapping of the vial) at RT overnight.

TABLE 11
Reagents used for the repeat experiments using (R)-Trolox
and the 4 bases that previously produced salts with (R)-
Trolox but did not produce salts with (S)-Trolox

| Reagent | | MW | density | mmol | equi | amount | Supplier | Solvent |
|---|---|---|---|---|---|---|---|---|
| R-trolox | | 250.29 | n/a | 0.400 | 1 | 100.0 | JMPS | N/A |
| 4 | (R)-(+)-2- | 151.21 | n/a | 0.420 | 1.05 | 63.4 | Aldrich | IPAc |
| | Amino-3- | | | | | | | |
| | phenyl-1- | | | | | | | |
| | propanol | | | | | | | |
| 7 | N-methyl-D- | 195.21 | n/a | 0.420 | 1.05 | 81.9 | Alfa | 5% |
| | glucamine | | | | | | Aesar | water/IPA |
| 9 | (1S,2S)-(+)- | 165.23 | n/a | 0.420 | 1.05 | 69.3 | Aldrich | IPAc |
| | Pseudoephedrine | | | | | | | |
| 10 | D-Lysine | 146.19 | n/a | 0.420 | 1.05 | 61.3 | Sigma | 5% |
| | | | | | | | | water/IPA |

TABLE 12
Results from the repeat experiments using (R)-Trolox and the 4 bases that previously
produced salts with (R)-Trolox but did not produce salts with (S)-Trolox

| | | | | | | | Comparison |
|---|---|---|---|---|---|---|---|
| | | | | After | | | to previous |
| | | In solvent | In solvent | cooling | | | R-Trolox salt |
| Exp | Base | at RT | at 40° C. | down | XRPD | On Plate | XRPD |
| 4 | 4 | bright | white | white | crystalline | white | same |
| | | yellow | solids, not | solids, not | salt | solid | polymorph |
| | | solution | stirring | stirring | | | |
| 7 | 7 | base did not | yellow | white | crystalline | white | same |
| | | dissolve | solution | solids, not | salt | solid | polymorph |
| | | | | stirring | | | |
| 9 | 9 | yellow | white | white | crystalline | white | different |
| | | solution | solids, not | solids, not | salt | solid | polymorph |
| | | | stirring* | stirring | | | |
| 10 | 10 | base did not | thin yellow | thin yellow | crystalline | white | slightly |
| | | dissolve | slurry | slurry** | salt | solid | different |
| | | | | | | | polymorph |

*The first time, the solid precipitated after the addition of diethyl ether. This time, the solid was produced after stirring in the solvent at 40° C. The two solids produced at different times are different polymorphs as suggested by XRPD data
**The first time, the solid was isolated after cooling down over the weekend. The second time, the solid was isolated after cooling down overnight and XRPD data showed the presence of base only. Therefore, seed from the previous experiment (Example 2) was added to the vial and the solution was stirred. After a few hours, the solid was isolated but again XRPD data showed the presence of base only. The solution was then allowed to stir over the weekend. The solid isolated after the weekend was a slightly different polymorph than that obtained in Example 2 as suggested by XRPD data.

(R)-(+)-2-Amino-3-phenyl-1-propanol and N-methyl-D-glucamine produced the same polymorphs as the first time. (1S,2S)-(+)-Pseudoephedrine and D-Lysine produced different polymorphs from the first time.

### Example 6

### Salt Screening Experiments Using (S)-Trolox and Bases that Did Not Produce Salts with (R)-Trolox

(S)-Trolox (1.70 g) was weighed out in a vial and dissolved in ˜8.5 mL of EtOH. The total volume of this solution was measured out to be 9.6 mL. Therefore, 0.565 mL of this solution corresponded to 100 mg of (S)-Trolox. 1.05 eq. (100 mg of (S)-Trolox=1 eq.) of each base was weighed out in 15 different vials (3 vials per base). 0.565 mL of the (S)-Trolox solution was added to each vial and the solvent was evaporated slowly in a vacuum oven at RT. 0.500 mL of each solvent (a: IPAc, b: 2-MeTHF, and c: 5% water/IPA) was added to the appropriate vial and the solution was stirred at 40° C. for 1.5 hours using magnetic stirrer. Then, the solution was cooled down to RT for at least 1.5 hours. If solids precipitated, they were filtered and analyzed by XRPD and optical microscopy. If no solids precipitated, the solvent was evaporated slowly (uncapping of the vial) at RT overnight. If solids were obtained after evaporation, they were analyzed by XRPD and optical microscopy. If no solids were obtained after evaporation, 0.500 mL of diethyl ether was added to gel in the vial and stirred. If solids precipitated, they were filtered and analyzed by XRPD and optical microscopy. If no solids precipitated, the solvent was evaporated slowly (uncapping of the vial) at RT overnight.

TABLE 13
Reagents used for the salt screening experiments using (S)-
Trolox and bases that did not produce salts with (R)-Trolox

| Reagent | | MW | density | mmol | equi | amount | Supplier |
|---|---|---|---|---|---|---|---|
| S-trolox | | 250.29 | n/a | 0.400 | 1 | 100.0 | JMPS |
| 11 | L-Lysine | 146.19 | n/a | 0.420 | 1.05 | 61.3 | Aldrich |
| 12 | L-Histidine | 155.15 | n/a | 0.420 | 1.05 | 65.1 | Alfa |
| | | | | | | | Aesar |
| 13 | L-Arginine | 174.20 | n/a | 0.420 | 1.05 | 73.1 | Alfa |
| | | | | | | | Aesar |
| 15 | (R)-(−)- | 117.19 | 0.9 | 0.420 | 1.05 | 54.6 | Alfa |
| | Leucinol | | | | | | Aesar |
| 17 | (R)-(−)-2- | 75.11 | 0.963 | 0.420 | 1.05 | 32.7 | Alfa |
| | Amino-1- | | | | | | Aesar |
| | propanol | | | | | | |

In the following tables, (a) refers to Isopropyl acetate (IPAc), (b) refers to 2-methyltetrahydrofuran (2-MeTHF), and (c) refers to 5% water/isopropyl alcohol (v/v).

TABLE 14
Results from the salt screening experiments using (S)-Trolox
and bases that did not produce salts with (R)-Trolox

| | | | | | | After |
|---|---|---|---|---|---|---|
| | | | After | | After | evaporation |
| | | | evaporation | In solvent | cooling | from solvent |
| Exp | Base | In EtOH | from EtOH | a/b/c at 40° C. | down | a/b/c |
| 11a | 11 | base did not | dark yellow | yellow sticky | yellow sticky | N/A |
| | | dissolve | gel | material in | material in | |
| | | | | clear liquid | clear liquid | |
| 11b | | base did not | dark yellow | yellow sticky | thin yellow | N/A |
| | | dissolve | gel | material in | slurry | |
| | | | | clear liquid | | |
| 11c | | base did not | dark yellow | cloudy yellow | cloudy yellow | N/A |
| | | dissolve | gel | solution | solution | |
| 12a | 12 | base did not | off white | off white | off white | N/A |
| | | dissolve | solid | slurry | slurry | |
| 12b | | base did not | off white | off white | off white | N/A |
| | | dissolve | solid | slurry | slurry | |
| 12c | | base did not | off white | off white | off white | N/A |
| | | dissolve | solid | slurry | slurry | |
| 13a | 13 | base did not | white solid | white solid | white solid | N/A |
| | | dissolve | in yellow | chunks in clear | chunks in clear | |
| | | | gel | liquid | liquid | |
| 13b | | base did not | white solid | white solid, | white solid, not | N/A |
| | | dissolve | in yellow | not stirring | stirring | |
| | | | gel | | | |
| 13c | | base did not | white solid | thin yellow | yellow sticky | N/A |
| | | dissolve | in yellow | slurry | material in | |
| | | | gel | | clear liquid | |
| 15a | 15 | yellow | dark yellow | yellow | yellow | orange gel |
| | | solution | gel | solution | solution | |
| 15b | | yellow | dark yellow | yellow | yellow | orange waxy |
| | | solution | gel | solution | solution | solid |
| 15c | | yellow | dark yellow | yellow | yellow | orange gel |
| | | solution | gel | solution | solution | |
| 17a | 17 | yellow | dark yellow | yellow | yellow | orange gel |
| | | solution | gel | solution | solution | |
| 17b | | yellow | dark yellow | yellow | yellow | orange gel |
| | | solution | gel | solution | solution | |
| 17c | | yellow | dark yellow | yellow | yellow | orange gel |
| | | solution | gel | solution | solution | |

TABLE 15
Results from the salt screening experiments using (S)-Trolox and
bases that did not produce salts with (R)-Trolox (continued)

| | | | After | | | |
|---|---|---|---|---|---|---|
| | | After | evaporation | After stirring in | | |
| | | addition of | from diethyl | solvent a/b/c over | | |
| Exp | Base | diethyl ether | ether | the weekend | XRPD | On Plate |
| 11a | 11 | N/A | N/A | off white slurry with | crystalline | off white |
| | | | | sticky orange | salt | solid |
| | | | | material | | |
| 11b | | N/A | N/A | off white slurry | crystalline | waxy solid |
| | | | | | salt | |
| 11c | | yellow sticky | N/A | yellow sticky | no solid | N/A |
| | | material in | | material in clear | | |
| | | clear liquid* | | liquid | | |
| 12a | 12 | N/A | N/A | off white slurry | base | white solid |
| 12b | | N/A | N/A | off white slurry | base | white solid |
| 12c | | N/A | N/A | off white slurry** | base | white solid |
| 13a | 13 | N/A | N/A | white solid chunks in | amorphous | waxy solid |
| | | | | clear liquid | | |
| 13b | | N/A | N/A | N/A | crystalline | waxy solid |
| | | | | | salt | |
| 13c | | N/A | N/A | yellow sticky | no solid | N/A |
| | | | | material in clear | | |
| | | | | liquid | | |
| 15a | 15 | off white | N/A | N/A | crystalline | waxy solid |
| | | thick slurry | | | salt | |
| 15b | | N/A | N/A | N/A | crystalline | waxy solid |
| | | | | | salt | |
| 15c | | yellow | orange gel | N/A | no solid | N/A |
| | | solution | | | | |
| 17a | 17 | yellow sticky | N/A | N/A | no solid | N/A |
| | | material in | | | | |
| | | clear liquid | | | | |
| 17b | | yellow sticky | N/A | N/A | no solid | N/A |
| | | material in | | | | |
| | | clear liquid | | | | |
| 17c | | yellow sticky | N/A | N/A | no solid | N/A |
| | | material in | | | | |
| | | clear liquid | | | | |

*Since was a cloudy yellow solution after cooling down (see Table 14), 0.250 mL of diethyl ether was added to the vial as an anti-solvent. However, no solid precipitated and the result was a yellow sticky material in clear liquid.
**XRPD data showed the presence of base only. In order to help the base dissolve, 5% H₂O was added to the vial and the solution was allowed to stir over the weekend. Afterwards, XRPD data showed the presence of base only.

After initial XRPD screening, 3 crystalline salts were isolated and further analyzed by ¹H-NMR and optical microscopy. The promising bases that formed solid salts with (S)-Trolox but no solid with (R)-Trolox were L-Lysine, L-Arginine, and (R)-(−)-Leucinol.

Based on our salt screening experiments using (R) or (5) Trolox isomers, the following ranking table was developed. Preference was for bases that formed salts with only one isomer, and cost from lowest to highest. No changes were based on stoichiometry.

TABLE 16
Ranking table based on salt screening experiments using (R) or (S) Trolox isomers

| | | | | | | Ob- | | |
|---|---|---|---|---|---|---|---|---|
| | | Cost | R- | S- | | served | | |
| | | ($) | Trolox | Trolox | Mor- | Poly- | Stoichi- | |
| | Base | per g | Salt | Salt | phology | morphs | ometry | Process of Isolation |
| 7 | N-methyl-D- | 0.4 | yes | no | R salt: | 1 | 1 to 1 | frozen white solid after |
| | glucamine | | | | small to | | | cooling down |
| | | | | | large | | | |
| | | | | | crystals | | | |
| 13 | L-Arginine | 0.8 | no | yes | S salt: | 1 | 1 to 1.26 | frozen white solid after |
| | | | | | medium | | | cooling down |
| | | | | | sized | | | |
| | | | | | crystals | | | |
| 11 | L-Lysine | 2.4 | no | yes | S salt: | 1 | 1 to 1.17 | off white slurry after |
| | | | | | small to | | | cooling down |
| | | | | | large | | | over the weekend |
| | | | | | crystals | | | |
| 9 | (1S,2S)-(+)- | 8.6 | yes | no | R salt: | 2 | 1 to 1 | white slurry after |
| | Pseudo- | | | | large | | | addition of diethyl |
| | ephedrine | | | | crystals | | | ether or frozen |
| | | | | | or small | | | white solid after |
| | | | | | needle- | | | cooling down |
| | | | | | like | | | |
| | | | | | crystals | | | |
| 15 | (R)-(−)- | 30.6 | no | yes | S salt: | 2 | 1 to 1.06 | orange waxy solid |
| | Leucinol | | | | large | | | after evaporation |
| | | | | | crystals | | | from solvent or |
| | | | | | | | | off white thick |
| | | | | | | | | slurry after addition |
| | | | | | | | | of diethyl ether |
| 10 | D-Lysine | 40.8 | yes | no | R salt: | 2 | cannot be | off white slurry after |
| | | | | | medium | | determined | cooling down |
| | | | | | sized | | | over the weekend |
| | | | | | crystals | | | |
| 4 | (R)-(+)-2- | 43.0 | yes | no | R salt: | 1 | 1 to 1.19 | frozen white solid after |
| | Amino-3- | | | | small to | | | cooling down or |
| | phenyl-1- | | | | large | | | waxy solid after |
| | propanol | | | | needle- | | | evaporation from solvent |
| | | | | | like | | | |
| | | | | | crystals | | | |

### Example 7

### Chiral Resolution Experiments Using (R/S)-Trolox and the 7 Bases that Previously Produced Salts with (R)-Trolox or (S)-Trolox Only

(R/S)-Trolox (5.8 g) was weighed out in a vial and dissolved in ˜45 mL of EtOH. The total volume of this solution was measured out to be 49 mL. Therefore, 0.845 mL of this solution corresponded to 100 mg of (R/S)-Trolox. 1.05 eq. or 0.55 eq. (100 mg of (R/S)-Trolox=1 eq.) of each base was weighed out in different vials (for (R)-(+)-2-Amino-3-phenyl-1-propanol and (1S,2S)-(+)-Pseudoephedrine, a stock solution was made in methanol and the appropriate amount of the stock solution was added to different vials). Then, 0.845 mL of the (R/S)-Trolox solution was added to each vial and the solvent was evaporated slowly in a vacuum oven at RT.

0.500 mL of each solvent (a: IPAc, b: 2-MeTHF, c: 5% water/IPA, and d: EtOAc) was added to the appropriate vial and the solution was stirred at 40° C. for 1.5 hours using magnetic stirrer. Then, the solution was cooled down to RT for at least 1.5 hours. If solids precipitated, they were filtered and analyzed by XRPD and chiral HPLC. If no solids precipitated, the solvent was evaporated slowly (uncapping of the vial) at RT overnight. If solids were obtained after evaporation, they were analyzed by XRPD and chiral HPLC. If no solids were obtained after evaporation, 0.500 mL of diethyl ether was added to gel in the vial and stirred. If solids precipitated, they were filtered and analyzed by XRPD and chiral HPLC. If no solids precipitated, the solvent was evaporated slowly (uncapping of the vial) at RT overnight.

TABLE 17
Reagents used for the chiral resolution experiments using (R/S)-Trolox and the
7 bases that previously produced salts with (R)-Trolox or (S)-Trolox only

| Reagent | | MW | density | mmol | equi | amount | Supplier |
|---|---|---|---|---|---|---|---|
| RS-Trolox | | 250.29 | n/a | 0.400 | 1 | 100.0 | JMPS |
| 4 | (R)-(+)-2-Amino- | 151.21 | n/a | 0.420 | 1.05 | 63.4 | Aldrich |
| | 3-phenyl-1- | | | | | | |
| | propanol | | | | | | |
| | | | | 0.220 | 0.55 | 33.2 | |
| 7 | N-methyl-D- | 195.21 | n/a | 0.420 | 1.05 | 81.9 | Alfa |
| | glucamine | | | | | | Aesar |
| | | | | 0.220 | 0.55 | 42.9 | |
| 9 | (1S,2S)-(+)- | 165.23 | n/a | 0.420 | 1.05 | 69.3 | Aldrich |
| | Pseudoephedrine | | | | | | |
| | | | | 0.220 | 0.55 | 36.3 | |
| 10 | D-Lysine | 146.19 | n/a | 0.420 | 1.05 | 61.3 | Sigma |
| | | | | 0.220 | 0.55 | 32.1 | Sigma |
| 11 | L-Lysine | 146.19 | n/a | 0.420 | 1.05 | 61.3 | Aldrich |
| | | | | 0.220 | 0.55 | 32.1 | |
| 13 | L-Arginine | 174.20 | n/a | 0.420 | 1.05 | 73.1 | Alfa |
| | | | | | | | Aesar |
| | | | | 0.220 | 0.55 | 38.3 | |
| 15 | (R)-(−)-Leucinol | 117.19 | 0.9 | 0.420 | 1.05 | 54.6 | Alfa |
| | | | | | | | Aesar |
| | | | | 0.220 | 0.55 | 28.6 | |
| * | (R)-(+)-Alpha- | 121.18 | 0.952 | 0.420 | 1.05 | 53.4 | JMPS |
| | methylbenzylamine | | | | | | |
| | | | | 0.220 | 0.55 | 28.0 | |

In the following table, (a) refers to Isopropyl acetate (IPAc), (b) refers to 2-methyltetrahydrofuran (2-MeTHF), (c) refers to 5% water/isopropyl alcohol (v/v), and (d) refers to Ethyl acetate (EtOAc). Also, the second number (the first number being the base) corresponds to the equivalent amount: (1) refers to 0.55 equivalents and (2) refers to 1.05 equivalents.

TABLE 18
Results for the chiral resolution experiments using (R/S)-Trolox and the
7 bases that previously produced salts with (R)-Trolox or (S)-Trolox only

| | | | | | After | | |
|---|---|---|---|---|---|---|---|
| | | In solvent | After | After | evaporation | After | After |
| | | a/b/c/d at | cooling | stirring over | from solvent | addition | evaporation |
| Exp | Base | 40° C. | down | 2-3 days | a/b/c/d | of Et₂O | from Et₂O |
| 4a1 | 4 | yellow | yellow | light yellow | n/a | n/a | n/a |
| | | solution | solution, | slurry | | | |
| | | | add seed | | | | |
| | | | from | | | | |
| | | | Example 2 | | | | |
| | | | (4a) | | | | |
| 4a2 | 4 | yellow | light yellow | light yellow | n/a | n/a | n/a |
| | | solution | slurry | slurry | | | |
| 4b1 | 4 | yellow | yellow | yellow | orange | yellow | orange |
| | | solution | solution, | solution | gel with | solution | gel |
| | | | add seed | | some | | |
| | | | from | | solids | | |
| | | | Example 2 | | | | |
| | | | (4a) | | | | |
| 4b2 | 4 | yellow | yellow | yellow | orange | yellow | n/a |
| | | solution | solution, | solution | gel | slurry, | |
| | | | add seed | | | solid | |
| | | | from | | | sticks to | |
| | | | Example 2 | | | the sides | |
| | | | (4a) | | | | |
| 4c1 | 4 | yellow | yellow | yellow | solids in | yellow | orange |
| | | solution | solution, | solution | orange | solution | gel |
| | | | add seed | | gel | | |
| | | | from | | | | |
| | | | Example 2 | | | | |
| | | | (4a) | | | | |
| 4c2 | 4 | yellow | yellow | yellow | orange | light | n/a |
| | | solution | solution, | solution | gel | yellow | |
| | | | add seed | | | slurry | |
| | | | from | | | | |
| | | | Example 2 | | | | |
| | | | (4a) | | | | |
| 4d1 | 4 | yellow | yellow | yellow | solids in | thick | n/a |
| | | solution | solution, | solution | orange | light | |
| | | | add seed | | gel | yellow | |
| | | | from | | | slurry | |
| | | | Example 2 | | | | |
| | | | (4a) | | | | |
| 4d2 | 4 | yellow | light yellow | light yellow | n/a | n/a | n/a |
| | | solution | slurry | slurry | | | |
| 7a1 | 7 | sticky | sticky | sticky | n/a | n/a | n/a |
| | | material in | material in | material in | | | |
| | | clear liquid | clear liquid | clear liquid, | | | |
| | | | | white slurry | | | |
| | | | | after | | | |
| | | | | agitating | | | |
| | | | | with spatula | | | |
| 7a2 | 7 | sticky | sticky | sticky | n/a | n/a | n/a |
| | | material in | material in | material in | | | |
| | | clear liquid | clear liquid | clear liquid, | | | |
| | | | | white slurry | | | |
| | | | | after | | | |
| | | | | agitating | | | |
| | | | | with spatula | | | |
| 7b1 | 7 | frozen white | thick white | thick white | n/a | n/a | n/a |
| | | solid, add | slurry | slurry | | | |
| | | 500 μL | | | | | |
| 7b2 | 7 | cloudy | cloudy | cloudy | n/a | n/a | n/a |
| | | yellow | yellow | solution | | | |
| | | solution | solution | | | | |
| 7c1 | 7 | yellow | yellow | white slurry | n/a | n/a | n/a |
| | | solution | solution, | | | | |
| | | | add seed | | | | |
| | | | from | | | | |
| | | | Example 2 | | | | |
| | | | (7c) | | | | |
| 7c2 | 7 | sticky | yellow | white slurry | n/a | n/a | n/a |
| | | material in | solution, | | | | |
| | | yellow | add seed | | | | |
| | | solution | from | | | | |
| | | | Example 2 | | | | |
| | | | (7c) | | | | |
| 7d1 | 7 | sticky | sticky | white slurry | n/a | n/a | n/a |
| | | material in | material in | | | | |
| | | clear liquid | clear liquid | | | | |
| 7d2 | 7 | sticky | sticky | white slurry | n/a | n/a | n/a |
| | | material in | material in | | | | |
| | | clear liquid | clear liquid | | | | |
| 9a1 | 9 | white slurry | white slurry | white slurry | n/a | n/a | n/a |
| 9a2 | 9 | yellow | white slurry | white slurry | n/a | n/a | n/a |
| | | solution | | | | | |
| 9b1 | 9 | yellow | yellow | yellow | yellow | thick white | n/a |
| | | solution | solution, | solution | gel | slurry | |
| | | | add seed | | | | |
| | | | from | | | | |
| | | | Example 2 | | | | |
| | | | (9a) | | | | |
| 9b2 | 9 | yellow | yellow | yellow | yellow | white slurry, | n/a |
| | | solution | solution, | solution | gel | solid sticks to | |
| | | | add seed | | | the sides | |
| | | | from | | | | |
| | | | Example 2 | | | | |
| | | | (9a) | | | | |
| 9c1 | 9 | yellow | yellow | yellow | yellow | white slurry | n/a |
| | | solution | solution, | solution | gel | | |
| | | | add seed | | | | |
| | | | from | | | | |
| | | | Example 2 | | | | |
| | | | (9a) | | | | |
| 9c2 | 9 | yellow | yellow | yellow | yellow | yellow | yellow |
| | | solution | solution, | solution | gel | solution | gel |
| | | | add seed | | | | |
| | | | from | | | | |
| | | | Example 2 | | | | |
| | | | (9a) | | | | |
| 9d1 | 9 | yellow | yellow | white slurry | n/a | n/a | n/a |
| | | solution | solution, | | | | |
| | | | add seed | | | | |
| | | | from | | | | |
| | | | Example 2 | | | | |
| | | | (9a) | | | | |
| 9d2 | 9 | yellow | white slurry | white slurry | n/a | n/a | n/a |
| | | solution | | | | | |
| 10a1 | 10 | white slurry, | white slurry | white slurry | n/a | n/a | n/a |
| | | solid sticks | | | | | |
| | | to the sides | | | | | |
| 10a2 | 10 | white slurry, | white slurry, | white slurry, | n/a | n/a | n/a |
| | | solid sticks | solid sticks | solid sticks | | | |
| | | to the sides | to the sides | to the sides | | | |
| 10b1 | 10 | white slurry, | white slurry | white slurry | n/a | n/a | n/a |
| | | solid sticks | | | | | |
| | | to the sides | | | | | |
| 10b2 | 10 | solid chunks | solid chunks | solid chunks | n/a | n/a | n/a |
| | | in clear | in clear | in clear | | | |
| | | liquid | liquid | liquid | | | |
| 10c1 | 10 | thick white | thick white | thick white | n/a | n/a | n/a |
| | | slurry | slurry | slurry | | | |
| 10c2 | 10 | white slurry, | white slurry, | white slurry, | n/a | n/a | n/a |
| | | solid sticks | solid sticks | solid sticks | | | |
| | | to the sides | to the sides | to the sides | | | |
| 10d1 | 10 | white slurry, | white slurry | white slurry | n/a | n/a | n/a |
| | | solid sticks | | | | | |
| | | to the sides | | | | | |
| 10d2 | 10 | white slurry, | white slurry, | white slurry, | n/a | n/a | n/a |
| | | solid sticks | solid sticks | solid sticks | | | |
| | | to the sides | to the sides | to the sides | | | |
| 11a1 | 11 | white slurry, | white slurry, | white slurry, | n/a | n/a | n/a |
| | | solid sticks | solid sticks | solid sticks | | | |
| | | to the sides | to the sides | to the sides | | | |
| 11a2 | 11 | white slurry, | white slurry | white slurry | n/a | n/a | n/a |
| | | solid sticks | | | | | |
| | | to the sides | | | | | |
| 11b1 | 11 | sticky | sticky | sticky | n/a | n/a | n/a |
| | | material in | material in | material in | | | |
| | | clear liquid | clear liquid | clear liquid | | | |
| 11b2 | 11 | sticky | sticky | sticky | n/a | n/a | n/a |
| | | material in | material in | material in | | | |
| | | clear liquid | clear liquid | clear liquid | | | |
| 11c1 | 11 | sticky | thick white | thick white | n/a | n/a | n/a |
| | | material in | slurry | slurry | | | |
| | | clear liquid, | | | | | |
| | | white slurry | | | | | |
| | | after | | | | | |
| | | agitating | | | | | |
| | | with spatula | | | | | |
| 11c2 | 11 | sticky | thick white | thick white | n/a | n/a | n/a |
| | | material in | slurry | slurry | | | |
| | | clear liquid, | | | | | |
| | | frozen white | | | | | |
| | | solid after | | | | | |
| | | agitating | | | | | |
| | | with | | | | | |
| | | spatula, add | | | | | |
| | | 500 μL | | | | | |
| 13a1 | 13 | white slurry, | white slurry | white slurry | n/a | n/a | n/a |
| | | solid sticks | | | | | |
| | | to the sides | | | | | |
| 13a2 | 13 | white slurry, | white slurry | white slurry | n/a | n/a | n/a |
| | | solid sticks | | | | | |
| | | to the sides | | | | | |
| 13b1 | 13 | white solid | thin white | solid chunks | n/a | n/a | n/a |
| | | chunks in | slurry | in clear | | | |
| | | clear liquid | | liquid | | | |
| 13b2 | 13 | white slurry, | thin white | solid chunks | n/a | n/a | n/a |
| | | solid sticks | slurry | in clear | | | |
| | | to the sides | | liquid | | | |
| 13c1 | 13 | yellow | thick white | thick white | n/a | n/a | n/a |
| | | solution, | slurry | slurry | | | |
| | | thick white | | | | | |
| | | slurry after | | | | | |
| | | agitating | | | | | |
| | | with spatula | | | | | |
| 13c2 | 13 | yellow | thick white | thick white | n/a | n/a | n/a |
| | | solution | slurry | slurry | | | |
| | | with | | | | | |
| | | undissolved | | | | | |
| | | base, add | | | | | |
| | | 500 μL, | | | | | |
| | | white slurry | | | | | |
| 15a1 | 15 | thick white | thick white | thick white | n/a | n/a | n/a |
| | | slurry | slurry | slurry | | | |
| 15a2 | 15 | yellow | yellow | yellow | orange | yellow | n/a |
| | | solution | solution, | solution | gel | oil in | |
| | | | add seed | | | cloudy | |
| | | | from | | | solution | |
| | | | Example 2 | | | | |
| | | | (15a) | | | | |
| 15b1 | 15 | yellow | yellow | yellow | orange | thick white | n/a |
| | | solution | solution, | solution | gel with | slurry | |
| | | | add seed | | some | | |
| | | | from | | solids | | |
| | | | Example 2 | | | | |
| | | | (15a) | | | | |
| 15b2 | 15 | yellow | yellow | yellow | yellow | yellow | n/a |
| | | solution | solution, | solution | gel | oil in | |
| | | | add seed | | | cloudy | |
| | | | from | | | solution | |
| | | | Example 2 | | | | |
| | | | (15a) | | | | |
| 15c1 | 15 | yellow | yellow | yellow | white | frozen | n/a |
| | | solution | solution, | solution | solids | white | |
| | | | add seed | | | solid, add | |
| | | | from | | | 500 μL | |
| | | | Example 2 | | | | |
| | | | (15a) | | | | |
| 15c2 | 15 | yellow | yellow | yellow | yellow | yellow | yellow |
| | | solution | solution, | solution | gel | solution | gel |
| | | | add seed | | | | |
| | | | from | | | | |
| | | | Example 2 | | | | |
| | | | (15a) | | | | |
| *a1 | * | thick white | white slurry | white slurry | n/a | n/a | n/a |
| | | slurry | | | | | |
| *a2 | * | thick white | white slurry | white slurry | n/a | n/a | n/a |
| | | slurry | | | | | |
| *b1 | * | yellow | cloudy | white slurry | n/a | n/a | n/a |
| | | solution | yellow | | | | |
| | | | solution | | | | |
| *b2 | * | cloudy | white slurry | white slurry | n/a | n/a | n/a |
| | | yellow | | | | | |
| | | solution | | | | | |
| *c1 | * | cloudy | white slurry | white slurry | n/a | n/a | n/a |
| | | yellow | | | | | |
| | | solution | | | | | |
| *c2 | * | white slurry | white slurry | white slurry | n/a | n/a | n/a |

TABLE 19
Additional results for the chiral resolution experiments using (R/S)-Trolox and
the 7 bases that previously produced salts with (R)-Trolox or (S)-Trolox only

| | | | | Comparison to | | |
|---|---|---|---|---|---|---|
| | | | | previous salt | | |
| Exp | Base | XRPD | On Plate | XRPD | % ee (R) | er (R:S) |
| 4a1 | 4 | crystalline | white solid | same polymorph as | >99.9 | >99.9:0.1 |
| | | salt | | Example 2 (4a) | | |
| 4a2 | 4 | crystalline | white solid | same polymorph as | 99.5 | 99.8:0.2 |
| | | salt | | Example 2 (4a) | | |
| 4b1 | 4 | no solid | n/a | n/a | n/a | n/a |
| 4b2 | 4 | crystalline | white solid | same polymorph as | 44.0 | 72.0:28.0 |
| | | salt | | Example 2 (4a) | | |
| 4c1 | 4 | no solid | n/a | n/a | n/a | n/a |
| 4c2 | 4 | crystalline | white solid | new polymorph | racemic | racemic |
| | | salt | | | | |
| 4d1 | 4 | crystalline | white solid | same polymorph as | 99.1 | 99.6:0.4 |
| | | salt | | Example 2 (4a) | | |
| 4d2 | 4 | crystalline | white solid | same polymorph as | >99.9 | >99.9:0.1 |
| | | salt | | Example 2 (4a) | | |
| 7a1 | 7 | crystalline | white solid | new polymorph | racemic | racemic |
| | | salt | | | | |
| 7a2 | 7 | crystalline | white solid | new polymorph | racemic | racemic |
| | | salt | | | | |
| 7b1 | 7 | crystalline | white solid | new polymorph | racemic | racemic |
| | | salt | | | | |
| 7b2 | 7 | no solid | n/a | n/a | n/a | n/a |
| 7c1 | 7 | crystalline | white solid | new polymorph | racemic | racemic |
| | | salt | | | | |
| 7c2 | 7 | crystalline | waxy solid | same polymorph as | unable to | unable to |
| | | salt | | Example 2 (7c) | determine | determine |
| 7d1 | 7 | crystalline | white solid | new polymorph | racemic | racemic |
| | | salt | | | | |
| 7d2 | 7 | crystalline | white solid | new polymorph | racemic | racemic |
| | | salt | | | | |
| 9a1 | 9 | crystalline | white solid | new polymorph | racemic | racemic |
| | | salt | | | | |
| 9a2 | 9 | crystalline | white solid | same polymorph as | >99.9 | >99.9:0.1 |
| | | salt | | Example 2 (9a) | | |
| 9b1 | 9 | crystalline | white solid | new polymorph | racemic | racemic |
| | | salt | | | | |
| 9b2 | 9 | crystalline | white solid | same polymorph as | 61.8 | 80.9:19.1 |
| | | salt | | Example 2 (9a) | | |
| 9c1 | 9 | crystalline | white solid | new polymorph | racemic | racemic |
| | | salt | | | | |
| 9c2 | 9 | no solid | n/a | n/a | n/a | n/a |
| 9d1 | 9 | crystalline | white solid | new polymorph | racemic | racemic |
| | | salt | | | | |
| 9d2 | 9 | crystalline | white solid | same polymorph as | >99.9 | >99.9:0.1 |
| | | salt | | Example 2 (9a) | | |
| 10a1 | 10 | crystalline | white solid to | new polymorph | racemic | racemic |
| | | salt | waxy solid | | | |
| 10a2 | 10 | crystalline | white solid | new polymorph | racemic | racemic |
| | | salt | | | | |
| 10b1 | 10 | no solid | n/a | n/a | n/a | n/a |
| 10b2 | 10 | base never | n/a | n/a | n/a | n/a |
| | | dissolved | | | | |
| 10c1 | 10 | crystalline | white solid | new polymorph | racemic | racemic |
| | | salt | | | | |
| 10c2 | 10 | crystalline | white solid | new polymorph | racemic | racemic |
| | | salt | | | | |
| 10d1 | 10 | crystalline | white solid to | new polymorph | racemic | racemic |
| | | salt | waxy solid | | | |
| 10d2 | 10 | crystalline | white solid | new polymorph | racemic | racemic |
| | | salt | | | | |
| 11a1 | 11 | crystalline | white solid to | new polymorph | racemic | racemic |
| | | salt | waxy solid | | | |
| 11a2 | 11 | crystalline | white solid to | new polymorph | racemic | racemic |
| | | salt | waxy solid | | | |
| 11b1 | 11 | no solid | n/a | n/a | n/a | n/a |
| 11b2 | 11 | no solid | n/a | n/a | n/a | n/a |
| 11c1 | 11 | crystalline | white solid to | similar polymorph | racemic | racemic |
| | | salt | waxy solid | to Example 3 (11a) | | |
| 11c2 | 11 | crystalline | white solid to | similar polymorph | racemic | racemic |
| | | salt | waxy solid | to Example 3 (11a) | | |
| 13a1 | 13 | crystalline | white solid to | new polymorph | racemic | racemic |
| | | salt | waxy solid | | | |
| 13a2 | 13 | amorphous | white solid to | n/a | racemic | racemic |
| | | | waxy solid | | | |
| 13b1 | 13 | n/a | large crystals | n/a | n/a | n/a |
| | | | to oil | | | |
| 13b2 | 13 | n/a | white solid to | n/a | n/a | n/a |
| | | | oily solid | | | |
| 13c1 | 13 | amorphous | white solid | n/a | −36.7 | 31.7:68.3 |
| 13c2 | 13 | crystalline | white solid | new polymorph | −18.8 | 40.6:59.4 |
| | | salt | | | | |
| 15a1 | 15 | crystalline | white solid | new polymorph | racemic | racemic |
| | | salt | | | | |
| 15a2 | 15 | no solid | n/a | n/a | n/a | n/a |
| 15b1 | 15 | crystalline | white solid | new polymorph | racemic | racemic |
| | | salt | | | | |
| 15b2 | 15 | no solid | n/a | n/a | n/a | n/a |
| 15c1 | 15 | crystalline | white solid | new polymorph | racemic | racemic |
| | | salt | | | | |
| 15c2 | 15 | no solid | n/a | n/a | n/a | n/a |
| *a1 | * | crystalline | white solid | undesired | racemic | racemic |
| | | salt | | polymorph | | |
| *a2 | * | crystalline | white solid | undesired | racemic | racemic |
| | | salt | | polymorph | | |
| *b1 | * | crystalline | white solid | undesired | racemic | racemic |
| | | salt | | polymorph | | |
| *b2 | * | crystalline | white solid | undesired | racemic | racemic |
| | | salt | | polymorph | | |
| *c1 | * | crystalline | white solid | undesired | racemic | racemic |
| | | salt | | polymorph | | |
| *c2 | * | crystalline | white solid | undesired | racemic | racemic |
| | | salt | | polymorph | | |

Solids were isolated from all 7 bases. XRPD and HPLC data suggested that two of the bases ((R)-(+)-2-Amino-3-phenyl-1-propanol and (1S,2S)-(+)-Pseudoephedrine) could successfully resolve racemic Trolox and produce the corresponding (R)-Trolox salts with up to >99.9% ee.

### Example 8

### Stress Test Experiments Using (R/S)-Trolox and the 2 Bases that Successfully Resolved (R/S)-Trolox, in the Presence of the Corresponding Undesired Polymorphs

1.05 eq. or 0.55 eq. (100 mg of (R/S)-Trolox=1 eq.) of Base #4 or Base #9 was weighed out in 8 different vials. (R/S)-Trolox (100 mg) was added to each vial. A seed of undesired polymorphs (from experiment 10, 4c2 or 9a1, respectively) was added to the vials containing Base #4 or Base #9, respectively.

0.500 mL of the appropriate solvent (IPAc or EtOAc) was added to the appropriate vial and the solution was stirred at 40° C. for 1.5 hours using magnetic stirrer. Then, the solution was cooled down to RT for at least 1.5 hours. The solids that precipitated were filtered and analyzed by XRPD and chiral HPLC.

TABLE 20
Reagents used for the stress test experiments using (R/S)-Trolox
and the 2 bases that successfully resolved (R/S)-Trolox, in
the presence of the corresponding undesired polymorphs

| Reagent | | MW | density | mmol | equi | amount | Supplier |
|---|---|---|---|---|---|---|---|
| RS-Trolox | | 250.29 | n/a | 0.400 | 1 | 100.0 | JMPS |
| 4 | (R)-(+)-2- | 151.21 | n/a | 0.420 | 1.05 | 63.4 | Aldrich |
| | Amino-3- | | | | | | |
| | phenyl-1- | | | | | | |
| | propanol | | | | | | |
| | | | | 0.220 | 0.55 | 33.2 | |
| 9 | (1S,2S)-(+)- | 165.23 | n/a | 0.420 | 1.05 | 69.3 | Aldrich |
| | Pseudoephedrine | | | | | | |
| | | | | 0.220 | 0.55 | 36.3 | |

In the following table, (a) refers to Isopropyl acetate (IPAc) and (d) refers to Ethyl acetate (EtOAc). Also, the second number (the first number being the base) corresponds to the equivalent amount: (1) refers to 0.55 equivalents and (2) refers to 1.05 equivalents.

TABLE 21
Results from the stress test experiments using (R/S)-Trolox and the 2 bases that successfully
resolved (R/S)-Trolox, in the presence of the corresponding undesired polymorphs

| | | In | | | | | | |
|---|---|---|---|---|---|---|---|---|
| | | solvent | After | | | Comparison to | | |
| | | a/d at | cooling | | On | previous salt | | |
| Exp | Base | 40° C. | down | XRPD | Plate | XRPD | % ee (R) | er (R:S) |
| 4a1 | 4 | white | white | crystalline | white | undesired | racemic | racemic |
| | | slurry | slurry | salt | solid | polymorph | | |
| 4a2 | 4 | white | white | crystalline | white | undesired | racemic | racemic |
| | | slurry | slurry | salt | solid | polymorph | | |
| 4d1 | 4 | white | white | crystalline | white | undesired | racemic | racemic |
| | | slurry | slurry | salt | solid | polymorph | | |
| 4d2 | 4 | white | white | crystalline | white | undesired | racemic | racemic |
| | | slurry | slurry | salt | solid | polymorph | | |
| 9a1 | 9 | cloudy | white | crystalline | white | undesired | racemic | racemic |
| | | solution | slurry | salt | solid | polymorph | | |
| 9a2 | 9 | white | white | crystalline | white | desired | 72.3 | 86.1:13.9 |
| | | slurry | slurry | salt | solid | polymorph | | |
| 9d1 | 9 | cloudy | white | crystalline | white | undesired | racemic | racemic |
| | | solution | slurry | salt | solid | polymorph | | |
| 9d2 | 9 | white | white | crystalline | white | desired | 75.8 | 87.9:12.1 |
| | | slurry | slurry | salt | solid | polymorph | | |

XRPD and HPLC data suggested that when using 1.05 equivalents of (1S,2S)-(+)-Pseudoephedrine, the desired polymorph (enantioenriched salt) could form even in the presence of the undesired polymorph (racemic salt).

Samples from 2 out of the 8 stress test experiments (previously setup) were tested after 5-6 weeks of slurrying by XRPD. These 2 experiments (9a2 and 9d2) had successfully resolved (R/S)-Trolox by forming a salt of R-Trolox and (1S,2S)-(+)-Pseudoephedrine in 2 different solvents (IPAc and EtOAc). Both experiments had been carried out in the presence of the undesired racemic salt and 1.05 equivalents of the base had been added. IPAc was used as the solvent in experiment 9a2 while EtOAc was used in experiment 9d2. After ˜5-6 weeks of slurrying the slurries were filtered, the wet cakes were washed with the respective solvents and were subsequently confirmed to be the salt of R-Trolox by XRPD. XRPD of the samples after 5-6 weeks of slurrying were similar to XRPD of the original samples from Table 21.

### Example 9

### Gravimetric Solubility Analysis of the R-Trolox (1S,2S)-(+)-Pseudoephedrine Salt in 12 Different Solvent Systems at Room Temperature

R-Trolox (1S,2S)-(+)-Pseudoephedrine salt was prepared by weighing out 2 g of (R/S)-Trolox and 1.05 equivalents of (1S,2S)-(+)-Pseudoephedrine in a glass vial. 10 ml of EtOAc was added and the solution was stirred at 40° C. for ˜1.5 hours and then cooled to room temperature and stirred overnight. Next morning the slurry was filtered and the wet cake was placed in a vacuum oven for drying at room temperature for ˜2 hours. The dried solids were confirmed to be the R-Trolox (1S,2S)-(+)-Pseudoephedrine salt by XRPD. This material was used for solubility analysis.

Gravimetric solubility analysis of the R-Trolox (1S,2S)-(+)-Pseudoephedrine salt in 12 different solvent systems at room temperature was carried out. ˜45 mg each of the salt was weighed into 12 vials and 1 ml of the respective solvents was added at room temperature. If the solids dissolved completely ˜100 mg of the salt was added to the vial. If these solids also dissolved completely the solubility was noted as >145 mg/ml.

The samples which did not dissolve were slurried overnight. After overnight slurrying, the samples were centrifuged and 0.5 ml of the supernatant was added to pre-weighed glass vials and evaporated in a vacuum oven at room temperature over the weekend. Based on the weight of the solids obtained after evaporation the solubility was calculated. Also, the wet cakes obtained after centrifugation were analyzed by XRPD to confirm that no transformation had taken place. As shown in Table 22, this salt is highly soluble in acetone, ethanol (EtOH) and THF and moderately soluble in 2-MeTHF, IPAc, IPA, EtOAc and 2-MeTHF/IPAc mixtures. XRPD patterns of the solids obtained after centrifugation were consistent with a reference pattern of the R-Trolox (1S,2S)-(+)-Pseudoephedrine salt.

TABLE 22
Gravimetric solubility analysis of the R-Trolox (1S,2S)-(+)-
Pseudoephedrine salt in 12 different solvent systems at room temperature

| Expt. No. | | |
|---|---|---|
| 1957-52- | Solvent | Solubility |
| 1 | 2-MeTHF | 26 mg/ml |
| 2 | IPAc | 11 mg/ml |
| 3 | IPA | 39 mg/ml |
| 4 | Acetone | >145 mg/ml |
| 5 | EtOH | >145 mg/ml |
| 6 | EtOAc | 16 mg/ml |
| 7 | 2-MeTHF:Water | >145 mg/ml (in 2-phase |
| | (95:5) | system) |
| 8 | IPAc:Water (95:5) | >145 mg/ml (in 2-phase |
| | | system) |
| 9 | EtOAc:Water (95:5) | >145 mg/ml (in 2-phase |
| | | system) |
| 10 | THF | >145 mg/ml |
| 11 | 2-MeTHF/IPAc (3:1) | 32 mg/ml |
| 12 | 2-MeTHF/IPAc (1:3) | 32 mg/ml |

### Example 10

### Crystallization Experiments of the R-Trolox (1S,2S)-(+)-Pseudoephedrine Salt in 3 Different Solvent Systems

To study the effect of solvent system on crystallization of the R-Trolox (1S,2S)-(+)-Pseudoephedrine salt, 3 experiments in EtOAc, IPAc and 2-MeTHF (1957-54-1, 2 and 3 respectively) were setup. ˜200 mg each of the (R/S)-Trolox and 1.05 equivalents of the (1S,2S)-(+)-Pseudoephedrine were weighed into a glass vial followed by addition of 1 ml of the respective solvents. The solutions were stirred at 40° C. for ˜1 hour followed by stirring at room temperature for ˜1 hour.

The slurries obtained in experiments with EtOAc and IPAc were filtered and the wet cake was washed with the respective solvents and dried in a vacuum oven at room temperature for ˜1 hour. The solids were then confirmed as the R-Trolox (1S,2S)-(+)-Pseudoephedrine salt by XRPD and analyzed by chiral HPLC. The results of the chiral HPLC indicated that the % ee of the R-enantiomer was 99.5% for the EtOAc sample and 99.1% for the IPAc sample.

No solids were obtained in the experiment with 2-MeTHF (possibly due to relatively higher solubility of the salt). The sample was cooled to 10° C. and stirred overnight however no solids were obtained. The results of all 3 experiments are shown in Table 23. XRPD patterns of the solids obtained in experiments 1957-54-1 and 2 were consistent with the reference pattern of the R-Trolox (1S,2S)-(+)-Pseudoephedrine salt.

TABLE 23
Crystallization experiments of the R-Trolox (1S,2S)-(+)-
Pseudoephedrine salt in 3 different solvent systems

| Expt. No. | | | |
|---|---|---|---|
| 1957-54- | Solvent | XRPD Result | HPLC Result |
| 1 | EtOAc | R-Trolox (1S,2S)- | 99.5% ee of |
| | | (+)-Pseudoephedrine | R-enantiomer |
| | | salt | |
| 2 | IPAc | R-Trolox (1S,2S)- | 99.1% ee of |
| | | (+)-Pseudoephedrine | R-enantiomer |
| | | salt | |
| 3 | 2-MeTHF | No Solids | N/A |

### Example 11

### Gravimetric Solubility Analysis of the R-Trolox (1S,2S)-(+)-Pseudoephedrine Salt in EtOAc and IPAc at 60° C. and 5° C.

Solubility of R-Trolox (1S,2S)-(+)-Pseudoephedrine salt was measured gravimetrically at 60° C. and 5° C. in EtOAc and IPAc. ˜20-65 mg of the salt was slurried in the respective solvents overnight at both temperatures. After overnight slurrying, the samples were centrifuged and the supernatant was added to pre-weighed glass vials and evaporated in a vacuum oven at room temperature overnight. Based on the weight of the solids obtained after evaporation the solubility was calculated. Also, the wet cakes obtained after centrifugation were analyzed by XRPD (only for the 60° C. samples) to confirm that no transformation had taken place. Table 24 contains the solubility data at 60° C. and 5° C. in EtOAc and IPAc.

TABLE 24
Gravimetric solubility analysis of the R-Trolox (1S,2S)-(+)-
Pseudoephedrine salt in EtOAc and IPAc at different temperatures

| Expt. No. | Solvent/Temperature | Solubility |
|---|---|---|
| 1957-58-1 | EtOAc/5° C. | 3.4 mg/ml |
| 1957-58-2 | IPAc/5° C. | 2.6 mg/ml |
| 1957-56-1 | EtOAc/60° C. | 24 mg/ml |
| 1957-56-2 | IPAc/60° C. | 19 mg/ml |

### Example 12

### Experiments to Study the Effect of Equivalents of (1S,2S)-(+)-Pseudoephedrine Added on Resolution of (R/S)-Trolox

To study the effect of equivalents of (1S,2S)-(+)-Pseudoephedrine added on resolution of (R/S)-Trolox, 5 experiments with 0.5, 0.65, 0.85, 1 and 1.25 equivalents of the counterion were setup (experiments 1957-57-3, 4, 5, 6 and 7 respectively). EtOAc was used as the solvent. ˜200 mg each of the (R/S)-Trolox and the respective equivalents of the (1S,2S)-(+)-Pseudoephedrine were weighed into a glass vial followed by addition of 1 ml of EtOAc. The solutions were stirred at 40° C. for ˜2 hours followed by stirring at room temperature for ˜2 hours.

The slurries obtained in experiments with 0.85, 1 and 1.25 equivalents of the counterion (1957-57-5, 6 and 7 respectively) were filtered and the wet cake was dried in a vacuum oven at room temperature for ˜2 hours. The solids were then confirmed as the R-Trolox (1S,2S)-(+)-Pseudoephedrine salt by XRPD. In these experiments, solids were obtained while stirring at 40° C.

No solids were obtained in experiments with 0.5 and 0.65 equivalents of the counterion (1957-57-3 and 4) after ˜2 hours of stirring at 40° C. and ˜4 hours of stirring at room temperature. Both samples were stirred at 5° C. overnight.

After overnight stirring at 5° C., sample 1957-57-3 (0.5 eq.) was still a clear solution however solids were obtained in sample 1957-57-4 (0.65 eq.). The slurry obtained in experiment 1957-57-4 was filtered and the wet cake was dried in a vacuum oven at room temperature for ˜2 hours. The solids were then confirmed as the R-Trolox (1S,2S)-(+)-Pseudoephedrine salt by XRPD.

Sample 1957-57-3 (0.5 eq.) which did not yield any solids even after overnight stirring at 5° C. was brought back to room temperature and seeded with racemic salt (1957-44-9a1) and stirred overnight. The sample precipitated out overnight and a small portion of the slurry was filtered and the wet cake was dried in a vacuum oven at room temperature for ˜2 hours. XRPD analysis revealed that the solids obtained were the racemic salt.

Additional (1S,2S)-(+)-Pseudoephedrine was added to the slurry obtained in experiment 1957-57-3 to increase the equivalents of the counterion added from 0.5 to 1 and the sample was stirred at room temperature over the weekend. Next, a portion of the slurry was filtered and the solids obtained were analyzed by XRPD. The analysis revealed that the solids had transformed from the racemic salt to the R-Trolox (1S,2S)-(+)-Pseudoephedrine salt on increasing the counterion equivalents from 0.5 to 1.

The results of experiments 1957-57-3 to 7 are shown in Table 25.

TABLE 25
Experiments to study the effect of equivalents of (1S,2S)-(+)-
Pseudoephedrine added on resolution of (R/S)-Trolox with EtOAc as solvent

| Expt. No. | Equivalents of | | |
|---|---|---|---|
| 1957-57- | counterion | XRPD Result | Comments |
| 3 | 0.5 | See comments | Did not crystallize at 5° C. Seeding with |
| | | | racemic salt led to crystallization of |
| | | | racemic salt. Addition of counterion to |
| | | | increase equivalents from 0.5 to 1 led to |
| | | | R-Trolox (1S,2S)-(+)-Pseudoephedrine |
| | | | salt being obtained. |
| 4 | 0.65 | R-Trolox (1S,2S)-(+)- | Crystallized at 5° C. |
| | | Pseudoephedrine salt | |
| 5 | 0.85 | R-Trolox (1S,2S)-(+)- | Crystallized at 40° C. |
| | | Pseudoephedrine salt | |
| 6 | 1 | R-Trolox (1S,2S)-(+)- | Crystallized at 40° C. |
| | | Pseudoephedrine salt | |
| 7 | 1.25 | R-Trolox (1S,2S)-(+)- | Crystallized at 40° C. |
| | | Pseudoephedrine salt | |

### Example 13

### DSC Analysis on R-Trolox (1S,2S)-(+)-Pseudoephedrine Salt

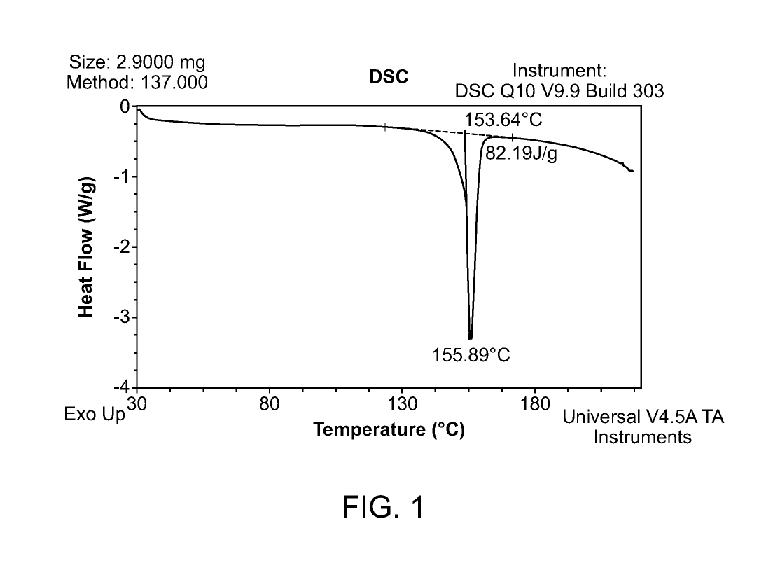

Sample 1957-57-6 was analyzed by DSC to obtain a reference thermogram for the R-Trolox (1S,2S)-(+)-Pseudoephedrine salt (FIG. 1).

### Example 14

### Resolution of (R/S)-Trolox at ˜5g Scale

Two experiments were carried out with 4 and 6 volumes (1957-61-3 and 4 respectively) of EtOAc as the solvent. In each experiment ˜4.75 g of (R/S)-Trolox and 2.67 g of (1S,2S)-(+)-Pseudoephedrine (0.85 eq.) were charged to a reactor at 40° C. and the respective volumes of EtOAc were added. The reactor was held at 40° C. for 1 hour, then cooled to 25° C. over 1 hour, followed by cooling to 10° C. over 30 minutes. Finally, the reactor was held at 10° C. for 1 hour.

The slurries obtained were filtered and the wet cake was washed with EtOAc (at 10° C.) and dried in a vacuum oven at room temperature over the weekend. Next, the solids obtained were weighed to calculate the yield and analyzed by XRPD and chiral HPLC. The results of both experiments are shown in Table 26. XRPD patterns of the solids obtained in experiments 1957-61-3 and 4 were consistent with the reference pattern of the R-Trolox (1S,2S)-(+)-Pseudoephedrine salt.

TABLE 26
Resolution of (R/S)-Trolox at ~5 g scale

| Expt. No. | Amount of | | | Chiral HPLC |
|---|---|---|---|---|
| 1957-61- | EtOAc | XRPD Result | Yield | Result |
| 3 | 4 volumes | R-Trolox (1S,2S)-(+)- | 66% | 97.4% ee |
| | | Pseudoephedrine salt | | |
| 4 | 6 volumes | R-Trolox (1S,2S)-(+)- | 62% | 97.8% ee |
| | | Pseudoephedrine salt | | |

Two experiments were carried out with 0.7 and 1 eq. (1957-63-1 and 2 respectively) of (1S,2S)-(+)-Pseudoephedrine and 5 volumes of EtOAc as the solvent. In each experiment ˜4.75 g of (R/S)-Trolox, the respective equivalents of (1S,2S)-(+)-Pseudoephedrine and 5 volumes of EtOAc were charged to a reactor at room temperature with 500 rpm overhead stirring. The reactor was heated to 40° C. over 30 minutes, held at 40° C. for 1 hour, cooled to 25° C. over 1 hour, followed by cooling to 10° C. over 30 minutes. Finally, the reactor was held at 10° C. for 1 hour.

Experiment 1957-63-1 (0.7 eq) was seeded (1%) with the (R)-Trolox-(1S,2S)-(+)-Pseudoephedrine salt to induce crystallization after no crystals were formed after 1 hour at 40° C. The slurries obtained in both experiments were filtered and the wet cake was washed with 1.5 volumes of EtOAc (at 10° C.) and dried in a vacuum oven at room temperature overnight. Next, the solids obtained were weighed to calculate the yield and analyzed by XRPD and chiral HPLC. The results of both experiments are shown in Table 27. XRPD patterns of the solids obtained in experiments 1957-63-1 and 2 were consistent with the reference pattern of the R-Trolox (1S,2S)-(+)-Pseudoephedrine salt.

Two experiments were carried out with 5 volumes of EtOAc and EtOAc with 1% water (1957-65-1 and 2 respectively) and 1.15 equivalents of (1S,2S)-(+)-Pseudoephedrine. In each experiment ˜4.75 g of (R/S)-Trolox, 1.15 equivalents of (1S,2S)-(+)-Pseudoephedrine and 5 volumes of the respective solvents were charged to a reactor at room temperature with 500 rpm overhead stirring. The reactor was heated to 40° C. over 30 minutes, held at 40° C. for 1 hour, cooled to 25° C. over 1 hour, followed by cooling to 10° C. over 30 minutes. Finally, the reactor was held at 10° C. for 1 hour.

The slurries obtained were filtered and a sample of the wet cake was saved for chiral HPLC analysis. The wet cake was washed with 6 volumes of EtOAc (at room temperature) and dried in a vacuum oven at room temperature overnight. Next, the solids obtained were weighed to calculate the yield and analyzed by XRPD and chiral HPLC. The results of both experiments are shown in Table 28. XRPD patterns of the solids obtained in experiments 1957-65-1 and 2 were consistent with the reference pattern of the R-Trolox (1S,2S)-(+)-Pseudoephedrine salt

TABLE 27
Resolution of (R/S)-Trolox at ~5 g scale

| Expt. | | | | Chiral | |
|---|---|---|---|---|---|
| No. | | | | HPLC | |
| 1957-63- | Counterion | XRPD Result | Yield | Result | Washing |
| 1 | 0.7 eq. | R-Trolox (1S,2S)- | 45% (Seeded to | 95.4% ee | 1.5 vols. |
| | | (+)-Pseudoephedrine | induce | | at 10° C. |
| | | salt | crystallization) | | |
| 2 | 1 eq. | R-Trolox (1S,2S)- | 73% | 98.0% ee | 1.5 vols. |
| | | (+)-Pseudoephedrine | | | at 10° C. |
| | | salt | | | |

TABLE 28
Resolution of (R/S)-Trolox at ~5 g scale

| Expt. No. | | | | Chiral HPLC | |
|---|---|---|---|---|---|
| 1957-65- | Solvent | XRPD Result | Yield | Result | Washing |
| 1 | EtOAc | R-Trolox (1S,2S)-(+)- | 65% | 99.9% ee | 6 vols. at |
| | | Pseudoephedrine salt | | (with washing) | Room temp |
| | | | | 91.6% | |
| | | | | (without washing) | |
| 2 | EtOAc with | R-Trolox (1S,2S)-(+)- | 60% | 99.9% ee | 6 vols. at |
| | 1% water | Pseudoephedrine salt | | (with washing) | Room temp |
| | | | | 94.3% | |
| | | | | (without washing) | |

### Example 15

### Slurry Experiments to Improve Enantiomeric Purity

Samples 1957-61-3, 4 and 1957-63-1, 2 (from pseudoephedrine salt formation experiments at ˜5g scale) were slurried in EtOAc at room temperature to try and improve their enantiomeric purity. ˜1 g of each of the samples was slurried in 2 volumes of EtOAc.

The slurries were sampled after 15 minutes and after 3 hours and were analyzed by chiral HPLC. The results of the analysis are shown in Table 29.

TABLE 29
Slurry Experiments to Improve Enantiomeric Purity

| | | | | EE by |
|---|---|---|---|---|
| | | | EE by Chiral HPLC | Chiral HPLC |
| Expt. No. | Original | Original | Result A | Result B |
| 1957-64- | Expt. | EE | (15 minutes slurry) | (3 hours slurry) |
| 1 | 1957-61-3 | 97.4% | 99.9% | 99.7% |
| 2 | 1957-61-4 | 97.8% | 99.9% | 99.8% |
| 3 | 1957-63-1 | 95.4% | 99.8% | 99.9% |
| 4 | 1957-63-2 | 98.0% | 99.9% | 99.9% |

### Example 16

### Process for Resolving (R)-Trolox

4.750 g of (R/S)-Trolox were added to reactor at room temperature. 1.15 equivalents (with respect to (R/S)-Trolox) of (1S,2S)-(+)-Pseudoephedrine (3.606 g) were added. 5 volumes of EtOAc (23.75 ml) were added. The reactor contents were heated to 40° C., the temperature held at 40° C. for 1 hour, then the reactor was cooled to 25° C. over 1 hour, then the reactor was cooled to 10° C. over 30 minutes, then the reactor was held at 10° C. for 1 hour. The slurry obtained was filtered, the wet cake washed with 6 volumes of EtOAc (28.5 ml) at room temperature, and the solids dried overnight in vacuum oven at 25-30° C. 2.569 g (65.2% yield) of (R)-Trolox-(1S,2S)-(+)-Pseudoephedrine salt (99.9% ee) was obtained.

### Example 17

### Synthesis of (R)-2-hydroxy-2-methyl-4-(2,4,5-trimethyl-3,6-dioxocyclohexa-1,4-dienyl)butanamide from (R)-Trolox

### Example 17A

### Extraction of (1S,2S)-(+)-Pseudoephedrine Free Base

To a suspension of (1S,2S)-(+)-Pseudoephedrine hydrochloride salt (300 g, Spectrum) in 2-MeTHF (1.5 L, 5 vol) was added 20% Aq NaOH solution (750 mL, 2.5 vol) and the mixture was stirred for 30 min (some solids remained undissolved) and transferred to a separatory funnel. The lower aqueous layer was drained along with solids that remained at the interphase and back extracted with 2-MeTHF (750 mL, 2.5 vol), the undissolved solids completely dissolved to form two clear layers. The combined organic layers were evaporated to dryness on rotavapor and the solids obtained were dried in a vacuum oven at 50° C. overnight to afford 240.3 g of free base as a white solid (97.7% recovery).

### Example 17B

### Precipitation of (1S,2S)-Pseudoephedrine from 2-MeTHF/heptane

(1S,2S)-pseudoephedrine (Sigma-Aldrich, sku #212464, 8.2 g) was dissolved at 50° C. in 2-MeTHF (41 ml, 5 vol). The resulting solution was diluted with heptane (82 ml, 10 vol) and the resulting suspension was stirred at room temperature overnight. The crystallized (1S,2S)-pseudoephedrine was filtered off and dried overnight at 40° C. under vacuum affording 6.4 g (78%) of white crystalline material. Filtrate was discarded to general waste.

Relatively low (78%) crystallization yield prompted an additional crystallization experiment with higher heptane to 2-MeTHF ratio. Crystalline (1S,2S)-pseudoephedrine obtained in the experiment above was dissolved at 50° C. in 2-MeTHF (32 ml, 5 vol). The resulting solution was diluted with heptane (32 ml, 5 vol) and the resulting suspension was chased with heptane (3×50 ml) on rotary evaporator until molar ration of 2-MeTHF to heptane became lower than 6% by NMR. The resulting suspension was filtered off and the product dried overnight at 40° under vacuum affording 6.3 g (98%) of white crystalline material.

### Example 17C

### Chiral Resolution of Trolox Using (1S,2S)-(+)-Pseudoephedrine

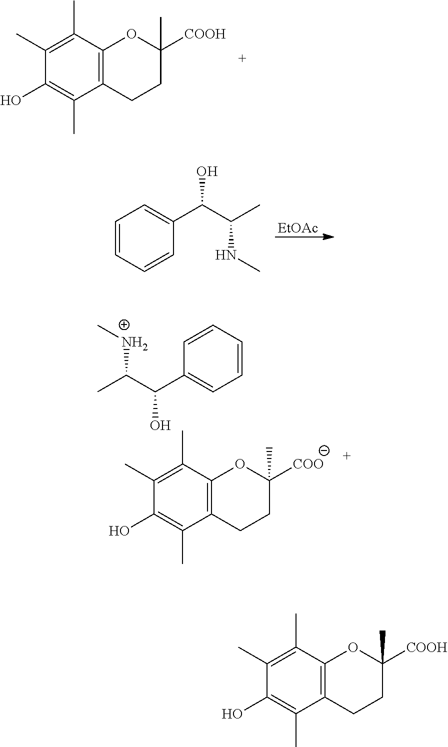

Racemic Trolox (316.6 g, 1.27 mol) and (1S,2S)-(+)-Pseudoephedrine free base described in Example 17A (240.0 g, 1.46 mol) were charged to a 4 L jacketed reactor equipped with an overhead stirrer, temperature probe and a nitrogen purge. Ethyl acetate (EtOAc, 1585 mL, 5 vol) was charged and the slurry was heated to 50° C. resulting in clear solution. (Premature (prior to complete dissolution of rac-trolox) precipitation of the (R)-trolox-pseudoephedrine salt ((R)-trolox-PE salt) was occasionally observed at 40° C. If premature precipitation takes place the reaction mixture was heated (usually to reflux temperature) to achieve complete dissolution.) The reaction mixture was cooled overnight to room temperature at which time massive precipitation was observed. The mixture was cooled to 10° C. over 30 min and held at this temperature for 1 h. The solids formed were collected by filtration, the wet cake was washed with EtOAc (1.9 L, 6 Vol) and the filter cake was dried in a vacuum oven at 25-30° C. to constant weight to afford 188.1 g (71.3% based on (R)-trolox) of a white solid. Chiral HPLC data indicated nearly 100% enantiomeric purity.

### Example 17D

### Recovery of (R)-Trolox from its Salt with (1S, 2S)-(+)-Pseudoephedrine

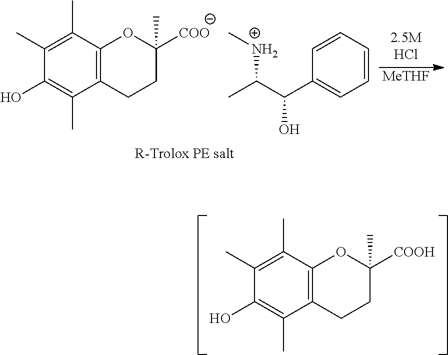

The resulting (R)-Trolox PE salt (187.3 g, 0.45 mol) was charged to a 2 L round-bottom flask followed by 2-MeTHF (570 ml, 3 vol.) to form a slurry. Hydrochloric acid (2.5 M, 325 ml, 0.81 mol, 1.75 eq) was added portionwise while maintaining temperature below 25° C. The (R)-trolox-PE salt was dissolved and (R)-Trolox was extracted into organic phase. Small black rag layer was observed in the interface and was kept with the aqueous. The aqueous phase was additionally extracted with 2-MeTHF (2×200 ml). The combined organic layer was then washed with 15% NaCl (200 ml) followed by water (200 ml). The organic layer was dried over anhydrous sodium sulfate (150 g), filtered and evaporated to dryness to afford white solid which was dried under vacuum oven at 30° C. to constant weight of 128.3 g, which is an overstoichiometric amount.

### Example 17E

### Preparation of (R)-6-hydroxy-2,5,7,8-tetramethylchromane-2-carboxamide

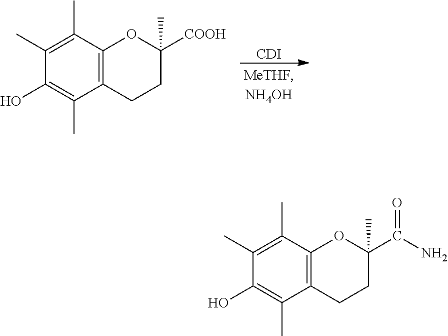

CDI (Sigma-Aldrich) (188 g, 1.16 mol) was charged to a 3-neck 2 L RBF equipped with an overhead stirrer, nitrogen inlet and temperature probe. 2-MeTHF (290 mL) was added to give a stirrable slurry followed by slow addition of (R)-trolox (126.0 g, 504 mmol) in 2-MeTHF (500 ml) at below 30° C. A slightly exothermic reaction accompanied by CO₂ evolution was observed. Outgassing started after addition of approximately one third of (R)-trolox. Complete dissolution of the starting materials was observed in approximately 15 min.

The content of this flask was slowly added to a pre-cooled to 5° C. 28-30% aqueous ammonia (380 ml) maintaining temperature below 30° C. The resulting biphasic suspension was stirred at room temperature and monitored by HPLC. The reaction was found to be complete at 36 h and was further processed after 48 h.

The reaction mixture was acidified to pH 1-2 with sulfuric acid (1:4 v/v) (850 ml) maintaining the temperature≦28° C., reaction was highly exothermic. The aqueous layer (pH=1) was removed and the organic layer was washed with NaCl (15% aqueous w/v, 250 mL), NaHCO₃ (1 M, 250 mL), NaCl (15% aqueous w/v, 250 mL) and water (250 ml). The majority of the organic layer was used for the subsequent steps.

### Example 17F

### Preparation of (R)-2-hydroxy-2-methyl-4-(2,4,5-trimethyl-3,6-dioxocyclohexa-1,4-dienyl)butanamide

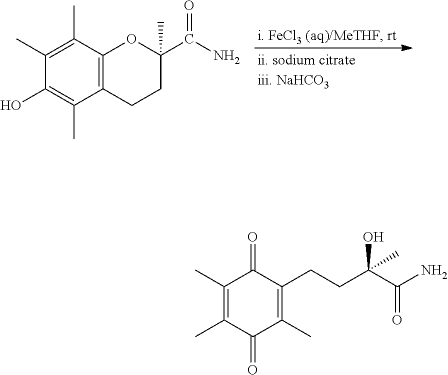

A solution of (R)-6-hydroxy-2,5,7,8-tetramethylchromane-2-carboxamide (708 ml) which contains ˜0.39 mole of the intermediate amide and water (126 ml) were charged to a 2 L 3N RBF equipped with an overhead stirrer and a thermocouple.

A stock solution of FeCl₃×6H₂O (480 g, 1.78 mol) in water (336 ml) was divided into 4 equal parts (204 g each) and one-fourth of the iron(III) chloride solution was added to the reaction flask. A weak (˜3° C.) exotherm was observed, the color of the organic layer turned nearly black then lightened to dark-brown. The biphasic reaction mixture was vigorously stirred for 40 min at room temperature. After removal of the lightly colored aqueous phase another portion of the iron(III) chloride solution was added and stirred for 40 min. The operation was repeated one more time and the organic phase was stored overnight at room temperature. The fourth treatment with FeCl₃×6H₂O was performed next morning. Nearly complete (99.44%) conversion of (R)-6-hydroxy-2,5,7,8-tetramethylchromane-2-carboxamide to (R)-2-hydroxy-2-methyl-4-(2,4,5-trimethyl-3,6-dioxocyclohexa-1,4-dienyl)butanamide was observed. Initial iron extraction was performed with 1M trisodium citrate solution (2×350 ml); the AUC % of (R)-6-hydroxy-2,5,7,8-tetramethylchromane-2-carboxamide increased to 0.84%. pH of the organic phase remained highly acidic (pH=1). A 1 ml aliquot of the organic phase was treated with 1M NaHCO₃ resulting in massive precipitation of red Fe(OH)₃. Based on this observation one more trisodium citrate wash (175 ml) was performed (0.74% (R)-6-hydroxy-2,5,7,8-tetramethylchromane-2-carboxamide). The repeat testing of the 1 ml aliquot with 1M NaHCO₃ gave no precipitation in the aqueous layer and the color of the aqueous layer was yellow, not red, indicating complete or nearly complete iron removal.

The organic layer was heated to 40° C. to prevent premature precipitation of (R)-2-hydroxy-2-methyl-4-(2,4,5-trimethyl-3,6-dioxocyclohexa-1,4-dienyl)butanamide and washed with 1M sodium bicarbonate solution (175 ml). The phase split was not immediate but was complete in 15 min forming two clear yellow layers. The organic layer (0.30% (R)-6-hydroxy-2,5,7,8-tetramethylchromane-2-carboxamide) was additionally washed with water (350 ml) giving 0.22% (R)-6-hydroxy-2,5,7,8-tetramethylchromane-2-carboxamide. Evaporation of the organic layer gave 96 g of (R)-2-hydroxy-2-methyl-4-(2,4,5-trimethyl-3,6-dioxocyclohexa-1,4-dienyl)butanamide.

The combined bicarbonate/water layers were back extracted with 2×250 ml of 2-MeTHF. Evaporation of these extracts separately gave 4.0 and 0.9 g of (R)-2-hydroxy-2-methyl-4-(2,4,5-trimethyl-3,6-dioxocyclohexa-1,4-dienyl)butanamide.

The combined solids (100.9 g—crude (R)-2-hydroxy-2-methyl-4-(2,4,5-trimethyl-3,6-dioxocyclohexa-1,4-dienyl)butanamide, 84% yield based on (R)-trolox-pseudoephedrine salt) were dissolved in isopropanol (600 ml) at 70° C. and the resulting yellow solution was charged to a 2 L 3N RBF equipped with an overhead stirrer a heating mantle and a thermocouple.

Heptane (600 ml) was added, no precipitation was observed. The reaction mixture was reheated to 55° C. and slowly cooled down to room temperature. Seeds of the desired polymorph (0.2 g) were added and the reaction mixture was stirred overnight at room temperature. Massive precipitation was observed overnight. The reaction mixture was cooled to 7° C. and stirred for additional 8 hours. The product was filtered, washed with isopropanol-heptane 1:1 v/v (2×75 ml) and dried over the weekend at 40° C. Yield 69.4 g of (R)-2-hydroxy-2-methyl-4-(2,4,5-trimethyl-3,6-dioxocyclohexa-1,4-dienyl)butanamide (58% based on (R)-trolox-pseudoephedrine salt).

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A method of separating Trolox isomers (R)-Trolox and (S)-Trolox, comprising:
(a) contacting a mixture of (R) and (S)-Trolox with a resolving agent selected from the group consisting of (1S,2S)-(+)-Pseudoephedrine, (R)-(+)-2-Amino-3-phenyl-1-propanol, (1R,2R)-(−)-Pseudoephedrine, and (S)-(+2-Amino-3-phenyl-1-propanol, wherein the resolving agent forms a solid salt with one of (R)-Trolox and (S)-Trolox, and substantially does not form a solid salt with the other; and
(b) separating the solid salt from the Trolox isomer that did not form the solid salt with the resolving agent.

2. The method of claim 1, wherein step (a) comprises dissolving the mixture of (R) and (S)-Trolox and the resolving agent in a solvent.

3.-9. (canceled)

10. The method of claim 2, wherein the solvent is ethyl acetate.

11.-14. (canceled)

15. The method of claim 2, wherein about 4 to about 6 volumes of solvent are added in step (a).

16.-18. (canceled)

19. The method of claim 1, wherein about 0.95 to about 1.20 equivalents of resolving agent are used in step (a).

20.-22. (canceled)

23. The method of claim 1, wherein the mixture in step (a) is seeded with the desired solid salt.

24. The method of claim 1, wherein step (b) comprises filtering the solid salt.

25. The method of claim 24, wherein step (b) further comprises a step (b)(1), comprising slurrying the solid salt in the solvent.

26. The method of claim 24, wherein step (b) further comprise rinsing and drying the solid salt.

27.-28. (canceled)

29. The method of claim 1, wherein the resolving agent is (1S,2S)-(+)-Pseudoephedrine.

30. The method of claim 1, wherein the resolving agent is (R)-(+)-2-Amino-3-phenyl-1-propanol.

31. (canceled)

32. The method of claim 1, comprising: (1) contacting a mixture of (R)-Trolox and (S)-Trolox with about 1.10 to about 1.20 equivalents of (1S,2S)-(+)-Pseudoephedrine and about 4 to about 6 volumes of ethyl acetate; (2) heating the mixture to between about 35° C. to about 55° C. until dissolution is achieved; (3) cooling the mixture to about 20° C. to about 30° C. over at least about 50 minutes; (4) cooling the mixture to about 5° C. to about 15° C. over about 20 to about 40 minutes; (5) holding the temperature in step (4) for about 50-70 minutes; (7) filtering the resulting slurry; (8) washing the wet cake with about 5 to about 7 volumes of ethyl acetate at room temperature; (9) and drying the solids.

33. (canceled)

34. The method of claim 1, wherein the enantiomeric excess of the (R)-Trolox obtained from the method is at least about 99%.

35.-36. (canceled)

37. The method claim 1, wherein the (R)-Trolox that is obtained from the method is converted to (R)-2-hydroxy-2-methyl-4-(2,4,5-trimethyl-3,6-dioxocyclohexa-1,4-dienyl)butanamide or the hydroquinone form thereof, or a salt thereof.

38.-43. (canceled)

44. A compound (R)-Trolox (1S,2S)-(+)-Pseudoephedrine salt or (R)-Trolox (R)-(+)-2-Amino-3-phenyl-1-propanol salt.

45. (canceled)

46. The method of claim 1, wherein the resolving agent is (1R,2R)-(−)-Pseudoephedrine.

47. The method of claim 1, wherein the resolving agent is (S)-(+2-Amino-3-phenyl-1-propanol.

48.-49. (canceled)

50. The method of claim 46, wherein the enantiomeric excess of the (S)-Trolox obtained from the method is at least about 99%.

51.-52. (canceled)

53. The method of claim 1, wherein the (S)-Trolox that is obtained from the method is converted to (S)-2-hydroxy-2-methyl-4-(2,4,5-trimethyl-3,6-dioxocyclohexa-1,4-dienyl)butanamide or the hydroquinone form thereof, or a salt thereof.

54.-59. (canceled)

60. A compound (S)-Trolox (1R,2R)-(−)-Pseudoephedrine salt or (S)-Trolox (S)-(−)-2-Amino-3-phenyl-1-propanol salt.

61. (canceled)
